(19) United States
(12) Patent Application Publication
(10) Pub. No.: US 2015/0064268 A1
(43) Pub. Date: Mar. 5, 2015

(54) NANOPARTICLES FOR STIMULATING ELASTOGENESIS

(71) Applicant: THE CLEVELAND CLINIC FOUNDATION, Cleveland, OH (US)

(72) Inventors: Anand Ramamurthi, Pepper Pike, OH (US); Balakrishnan Sivaraman, Westlake, OH (US)

(21) Appl. No.: 14/471,508

(22) Filed: Aug. 28, 2014

Related U.S. Application Data
(60) Provisional application No. 61/870,830, filed on Aug. 28, 2013.

(51) Int. Cl. A61K 31/14; A61K 9/51; A61K 41/00; A61K 31/65; A61K 47/48

(52) U.S. Cl. CPC A61K 31/14; A61K 31/65; A61K 47/48853; A61K 47/482; A61K 41/00; A61K 47/4893; A61K 9/51
USPC 424/501; 424/78.17; 424/400; 525/450; 525/54.1; 428/402

(57) ABSTRACT

Elastogenic nanoparticles including a polymeric core having a surface that is functionalized with a cationic amphiphilic compound, and comprising an active agent having pro-elastogenic and/or anti-proteolytic activity, are described herein. The elastogenic nanoparticles can be used in method of stimulating elastogenesis in a subject by administering to the subject a therapeutically effective amount of elastogenic nanoparticles.

### CROSS-REFERENCE TO RELATED APPLICATIONS

This application claims the benefit of U.S. Provisional Application No. 61/870,830, filed Aug. 28, 2013, which is incorporated herein by reference.

### GOVERNMENT FUNDING

This work was supported, at least in part, by award number HL092051 from the Department of Health and Human Services, National Institutes of Health. The United States government has certain rights in this invention.

### BACKGROUND

Abdominal aortic aneurysms or AAAs, the 13th leading cause of death in the United States, represent a disease condition which primarily afflicts senior males (>65 years of age). It manifests as a localized expansion/dilatation of the infrarenal aorta, due to progressive loss of elastin and/or elastic matrix from the aortic wall, on account of chronic overexpression of enzymes called matrix metalloproteases (MMPs)-2 and -9, which are elastolytic (i.e., they degrade elastin). As a result of this loss of elastin/elastic matrix, the vessel wall weakens and could potentially rupture fatally. Selle et al., Ann Surg., 189:158-64 (1979). Greater than 90% of detected AAAs are small (maximal diameter<5.5 cm), and grow very slowly (˜10% per year) over a period of 5 or more years. During this period, they are typically monitored via ultrasound or computed tomography (CT), until they attain a critical size of 5.5 cm, beyond which they are prone to rupture.

Upon reaching this critical size, AAAs are typically treated via open or (minimally invasive) endovascular repair (EVAR) techniques. However, these techniques have limitations on account of patient mortality and morbidity associated with open repair, as well as endoleaks and graft migration with EVAR which leads to continued blood flow into the aneurysm sac, potentially leading to rupture. Therefore, there is a need to develop non-surgical modalities for AAA therapy, as there are no established drug-based treatments for AAAs.

Non-surgical AAA therapies have focused on addressing (i) inhibition of MMPs and (ii) regeneration of elastin/elastic matrix, as both these phenomena are inter-related and are critical to the process of arrest and/or stabilization of AAA growth. MMP inhibition can be accomplished via pharmacological means using drugs, such as doxycycline (DOX), which has been shown to be effective in slowing growth in animal (Barton et al., Ann Vasc Surg., 20:228-36 (2006)) and human AAAs. (Mosorin et al., J Vasc Surg., 34:606-10 (2001)). Elastin regeneration on the other hand, is more complex, as elastic matrix assembly primarily occurs during development and is nearly complete by adolescence. On account of this, adult vascular smooth muscle cells (SMCs) have an inherently poor ability to synthesize elastin and assemble it into a mature fiber-based matrix, which compromises the ability of the vascular wall to auto-generate or repair elastic matrix following its proteolytic disruption or loss in AAA tissues.

Exogenous delivery of several different biomolecules and/or growth factors has been shown improve de novo elastin generation and assembly into a mature matrix. (Kothapalli et al. Tissue Eng., 15: 501-511 (2009); Gacchina et al. Tissue Eng., 17: 1945-1948 (2011); Gacchina et al. Tissue Eng., 17: 1699-1711 (2011); Sivaraman et al., Drug Deliv Transl Res., 2:323-50 (2012)). Their controlled spatio-temporal presentation is an important parameter in enhancing functional elastic matrix regeneration/generation and repair. Oral dosage of DOX, which leads to μg/mL range plasma concentrations of DOX has been shown to be effective in attenuating AAA growth by inhibiting MMPs-2 and -9 in both animal and human AAAs. However, this delivery modality has inherent limitations in DOX-dependent side effects, as well as the inhibition of de novo elastic matrix synthesis at DOX concentrations of 16-54 μg/mL, a critical step in AAA repair, by aortic SMCs.

Other groups have examined localized periaortic delivery of DOX from micropumps (Sho et al., J Vasc Surg., 39:1312-21 (2004)), which has functional benefits in terms of MMP-inhibition at the AAA site and concsequent suppression of AAA growth, with these effects being observed at delivered DOX doses equivalent to 1/100^{th} that of oral dosage. However, long-term pump implantation may have adverse effects in terms of promoting initiation of inflammation and adhesions within the surrounding tissue. Use of a DOX-loaded, controlled-release biodegradable fiber system as AAA therapy has also been described. (Yamawaki-Ogata et al., Biomaterials, 31:9554-64 (2010)). However, there remains a need for a more effective method of stimulating elastogenesis for treatment of AAA and a number of other conditions involving other tissues types (e.g., lung, dermis) that involve overexpression of MMPs and consequent elastin breakdown and loss.

### SUMMARY

The inventors have developed elastogenic nanoparticles for drug delivery, which are designed to enhance elastic matrix stabilization and regeneration, especially in proteolytic tissue environments.

The drug included in the nanoparticles may be any drug or biomolecule which has therapeutic benefits in regenerative medicine applications. The inventors' studies were directed to the use of elastogenic nanoparticles (NPs) to deliver doxycycline (DOX), a tetracycline derivative, which is an antibiotic. It has been shown to have effects in slowing AAA growth in animal models and in clinical studies, following oral dosage, via its ability to inhibit matrix metalloproteinases (MMPs)-2 and -9.

The elastogenic NPs of the invention can be used for localized, controlled, sustained DOX delivery. The route of delivery to AAAs may be via (i) endovascular or (ii) perivascular. Alternatively, these NPs may be integrated with other endovascular stents/grafts to stabilize the AAA wall and deliver other therapeutic agents that have pro-elastin regenerative and/or anti-proteolytic properties. In addition, surface functionalization of these NPs with cationic amphiphiles provides them with enhanced properties for stabilization and regeneration of elastic matrix, as well as MMP-inhibitory properties towards enhancing AAA repair.

### BRIEF DESCRIPTION OF THE FIGURES

FIG. 1 provides a schematic diagram for the NPs and their multifunctional effects on MMPs, as well as elastin synthesis and elastic matrix assembly processes in proteolytic tissue microenvironments.

FIG. 2 provides graphs showing the in vitro DOX release profiles from the PLGA NPs, at the three different DOX loadings (2%, 5% and 10% w:w ratio to PLGA), and the three NP concentrations of 0.2, 0.5 and 1.0 mg/mL. (n=3 per group; mean±SD).

FIG. 3 provides a bar graph showing the proliferation of aneurysmal SMCs (EaRASMCs) in response to DOX released from PLGA NPs at 0.2, 0.5 and 1.0 mg/mL NP concentrations. Healthy SMCs (RASMCs) were used as the cell-type control, while EaRASMCs treated with blank NPs (0% DOX) as the treatment control. The cell number was calculated based on an estimate of 6 pg of DNA per cell, via a DNA assay, at 0 and 21 days post-seeding, (mean±SD; n=3 per case, * denotes p<0.05 compared to untreated EaRASMCs; # denotes p<0.05 compared to 0% DOX-PLGA treatment controls).).

FIG. 4 provides bar graphs showing the effects of DOX released from PLGA NPs on (A) tropoelastin synthesis, and (B) synthesis of alkali-soluble and alkali-insoluble crosslinked matrix elastin by EaRASMCs. (C) Total matrix elastin was normalized to DNA content per cell. RASMCs were used as cell-type controls and EaRASMCs treated with blank (0% DOX) NPs as treatment control. (mean±SD; n=6/case for tropoelastin, n=3/case for tropoelastin, * denotes p<0.05 compared to untreated EaRASMCs; # denotes p<0.05 compared to 0% DOX-PLGA treatment controls). Note: Total matrix elastin for each well per test case was normalized to the average DNA content per test case.

FIG. 5 provides a gel image and bar graphs showing the effect of DOX released from NPs on MMP-2 synthesis and activity in EaRASMC cultures, as analyzed with western blots and gel zymography. (A) Representative image of western blot, with β-actin bands as loading controls. (B) Fold-change in production of active MMP-2 compared to control cultures without NPs. (C) Fold-change in MMP-2 activity compared to NP-untreated control EaRASMC cultures (representative zymogram in inset). Data represented (mean±SD; n=5/case for western blots, n=3/case for gel zymograms, * denotes p<0.05 compared to controls with no NPs, # denotes p<0.05 compared to 0% DOX-PLGA treatment controls). Notes: Values in FIG. 5B represent the fold-change in normalized band intensities of the active MMP-2 band for each treatment test case (normalized to its corresponding β-actin loading control band to enable accurate comparison between the different test cases) compared to untreated (no NP) controls. The normalized band intensity of MMP-2 for the NP-untreated control test condition was set to unity to determine fold-change in MMP-2 expression or activity for the different NP-treated test cases.

FIG. 6 provides a bar graph showing the percentage of PLGA NPs functionalized with the different amphiphilic surface-modifiers (with BSA-Cy5 encapsulated within) bound to elastin. (mean±SD; n=3/case, * denotes p<0.05 compared to corresponding amount of PVA-functionalized PLGA NPs) Note: The percentage retention was calculated after subtracting the amount of NPs adhering to empty wells.

FIG. 7 provides bar graphs showing the fold-change in lysyl oxidase (LOX) activity in EaRASMC cultures at 21 days, treated with the different surface-functionalized PLGA NPs. LOX activity was measured in (A) the pooled cell culture medium and (B) in the cell layer by the quantity of hydrogen peroxide released upon oxidative deamination of alkyl monoamines and diamines and normalized to that of the PVA-modified PLGA NP controls. (mean±SD; n=6/case; * denotes p<0.05 compared to PVA-functionalized PLGA NP controls).

FIG. 8 shows the effect of surface functionalization of PLGA NPs on MMP-2 synthesis and activity in EaRASMC cultures, as analyzed with western blots and gel zymography. (A) Representative image of western blot, with β-actin bands as loading controls. (B) Fold-change in production of active MMP-2 compared to control cultures without NPs. (C) Fold-change in MMP-2 activity compared to untreated control EaRASMC cultures. Data represented (mean±SD; n=3/case, * denotes p<0.05 compared to controls with no NPs). Notes: Values in FIG. 8B represent the fold-change in normalized band intensities of the active MMP-2 band for each treatment test case (normalized to its corresponding β-actin loading control band to enable accurate comparison between the different test cases) compared to NP-untreated controls. The normalized band intensity of MMP-2 for the NP-untreated control test condition was set to unity to determine fold-change in MMP-2 expression or activity for the different NP-treated test cases.

FIG. 9 provides a graph showing that co-incorporation of SPIONs within the polymer matrix of DOX-encapsulating PLGA NPs does not significantly alter their DOX release profiles or steady state release doses. Both sets of PLGA NPs were surface functionalized with DMAB, which imparted a positive surface charge. Shown are DOX release from PLGA NPs (0.5 mg/ml concentration), with and without co-encapsulated SPIONs (n=3; mean±SD). DOX release levels with both formulations were less than 5 μg/mL, which has been shown to limit EaRASMC proliferation and elastic matrix deposition.

FIG. 10 provides a bar graph showing that the lack of effect of DOX released from DMAB-surface functionalized PLGA NPs, on proliferation of rat aneurysmal smooth muscle cells (EaRASMCs), is maintained when the cells were cultured with NPs that contained both DOX and SPIONs. Plots show EaRASMC counts at 1 day (time controls), and at 21 days of culture with blank, DOX and DOX-SPION NPs (0.1 mg/mL concentration), with untreated EaRASMCs as control. n=6, mean±SD. * denotes p<0.05 vs. NP-untreated EaRASMC control. # denotes p<0.05 vs. blank NP control.

FIG. 11 provides bar graphs showing the effects of DOX-NPs and DOX-SPION NPs (0.1 mg/ml NP concentrations for both) on elastic matrix deposition by cultured EaRASMCs relative to cultures treated with blank NPs (active agent control; 0.1 mg/ml NP concentration) or cultured with no NPs (treatment control) on an (A) an absolute basis (left) and (B) DNA-normalized basis (right). Cationic, DMAB functionalized NPs caused a significant improvement in elastic matrix deposition versus NP-free cultures, as seen in all NP-treated cases. DOX release from the NPs further enhanced this effect. DOX release from NPs co-incorporating both DOX and SPIONs however did not induce similar further increases in elastic matrix deposition, likely due to slightly lower/slower release of DOX in the presence of SPIONs (FIG. 9). Data shown were obtained by analysis of n=6 replicate cultures, and values shown are mean±S.D. * denotes p<0.05 vs. NP-untreated EaRASMC control; # denotes p<0.05 vs. blank NP control.

FIG. 12 provides bar graphs showing the effects of DOX release from DMAB-functionalized PLGA NPs with and without co-encapsulated SPIONs (0.1 mg/ml NP concentration) on MMP synthesis (A; western blot) and activity (B; gel zymography) by EaRASMCs (n=3, mean±S.D. * denotes p<0.05 vs. NP-untreated EaRASMC control; # denotes p<0.05 vs. blank NP control. Data shows significant decreases in MMP2 protein synthesis and enzyme activity in both cases relative to NP-free cultures, and even versus blank NP-treated cultures. Despite the slightly lower release levels of DOX from the DOX-SPION NPs versus the DOX NPs, no significant differences in levels of attenuation of MMP2 were noted in cultures treated with the respective NP formulations. MMP-9 protein synthesis was too low for reliable quantification via densitometry, and hence excluded.

### DETAILED DESCRIPTION

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, and other references mentioned herein are incorporated by reference in their entirety.

### DEFINITIONS

As used in the description of the invention and the appended claims, the singular forms “a,” “an,” and “the” are intended to include the plural forms as well, unless the context clearly indicates otherwise. In addition, the recitations of numerical ranges by endpoints include all numbers subsumed within that range (e.g., 1 to 5 includes 1, 1.5, 2, 2.75, 3, 3.80, 4, 5, etc.).

As used herein, the terms “peptide,” “polypeptide” and “protein” are used interchangeably, and refer to a compound comprised of amino acid residues covalently linked by peptide bonds. A protein or peptide must contain at least two amino acids, and no limitation is placed on the maximum number of amino acids that can comprise the sequence of a protein or peptide. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. A protein may be a receptor or a non-receptor.

“Treating”, as used herein, means ameliorating the effects of, or delaying, halting or reversing the progress of a disease, injury process or disorder. The word encompasses reducing the severity of a symptom of a disease, injury process or disorder and/or the frequency of a symptom of a disease, injury process or disorder.

The language “effective amount” or “therapeutically effective amount” refers to a nontoxic but sufficient amount of the composition used in the practice of the invention that is effective to reduce, arrest, or restore healthy matrix state (including elastic matrix) and associated cell behavior in a subject. The desired treatment may be prophylactic and/or therapeutic. That result can be reduction and/or alleviation of the signs, symptoms, or causes of a disease or disorder, or any other desired alteration of a biological system. An appropriate therapeutic amount in any individual case may be determined by one of ordinary skill in the art using routine experimentation.

“Pharmaceutically acceptable carrier” refers herein to a composition suitable for delivering an active pharmaceutical ingredient, such as the composition of the present invention, to a subject without excessive toxicity or other complications while maintaining the biological activity of the active pharmaceutical ingredient. Protein-stabilizing excipients, such as mannitol, sucrose, polysorbate-80 and phosphate buffers, are typically found in such carriers, although the carriers should not be construed as being limited only to these compounds.

“Biocompatible” as used herein, refers to any material that does not cause injury or death to the animal or induce an adverse reaction in an animal when placed in intimate contact with the animal's tissues. Adverse reactions include for example inflammation, infection, fibrotic tissue formation, cell death, or thrombosis. The terms “biocompatible” and “biocompatibility” when used herein are art-recognized and mean that the referent is neither itself toxic to a host (e.g., an animal or human), nor degrades (if it degrades) at a rate that produces byproducts (e.g., monomeric or oligomeric subunits or other byproducts) at toxic concentrations, does not cause prolonged inflammation or irritation, or does not induce more than a basal immune reaction in the host. It is not necessary that any subject composition have a purity of 100% to be deemed biocompatible. Hence, a subject composition may comprise 99%, 98%, 97%, 96%, 95%, 90% 85%, 80%, 75% or even less of biocompatible agents, e.g., including polymers and other materials and excipients described herein, and still be biocompatible.

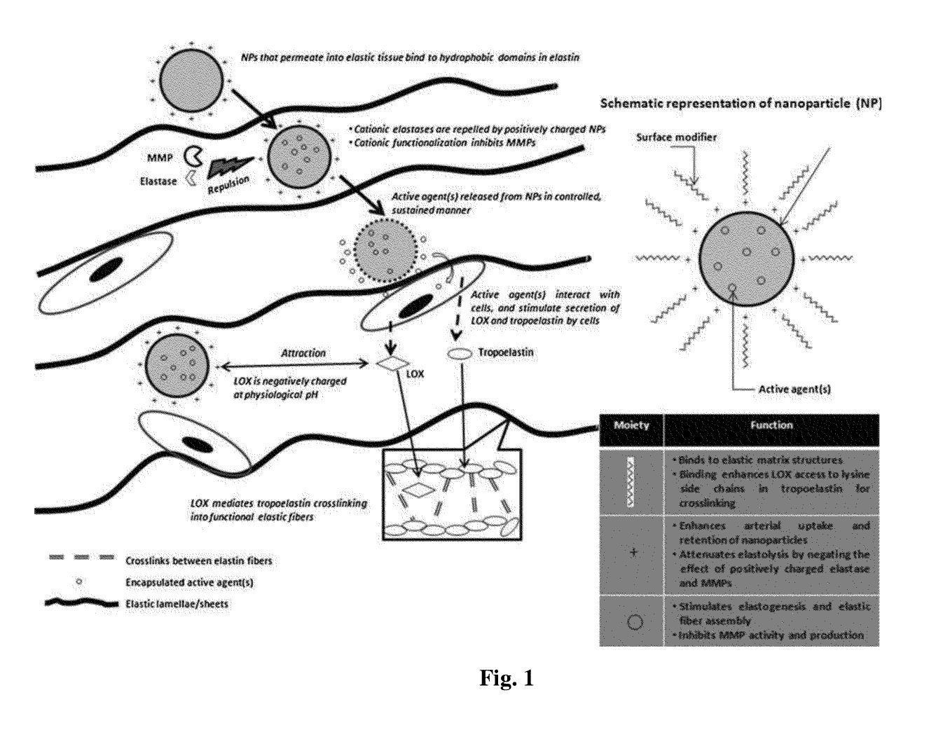

The invention provides multifunctional nanoparticles (NPs) for enhanced elastic matrix regenerative outcomes in tissue/matrix engineering. These NPs have two properties which are important for their use in regenerative therapies for AAA repair, as well as other diseases/conditions of vascular and other tissues which involve matrix (e.g., elastic) proteolysis. First, the NPs have been functionalized to include a cationic surface charge. Functionalized, as the term is used herein, refers to modification of the surface of the polymeric core of the NPs by attachment of a compound that varies the function of the NP. A cationic surface charge enhances permeation into tissues containing glycosaminoglycans (GAGs), such as the aortic wall, repels cationic elastases, inactivates MMPs via interactions with negatively charged glutamic acid residues in their active site, which are essential for MMP-activity, and provides electrostatic interactions with negatively charged lysyl oxidase (LOX; mediates crosslinking of elastin into mature elastic fibers) for enhanced functional fiber formation/deposition. Second, the NPs have been functionalized to have long hydrophobic alkyl chains. These long alkyl chains provide enhanced elastin/elastic matrix binding, due to interactions with hydrophobic domains in elastin and/or tissue, enhanced uptake and retention within the aortic wall, and binding to tropoelastin (via hydrophobic interactions) enhances access of lysyl oxidase (LOX) to lysine residues in tropoelastin, thereby enabling enhanced crosslinking of tropoelastin molecules into mature elastic matrix. Additionally, these long alkyl chains mediate inhibition of MMP activity via steric blockade of the active site of MMPs, thereby rendering it unable to recognize, bind and degrade ECM proteins, especially elastin. FIG. 1 provides a schematic diagram of the NPs and their effects.

In one aspect, the present invention provides an elastogenic nanoparticle comprising a polymeric core having a surface that is functionalized with a cationic amphiphilic compound. In some embodiments, the elastogenic nanoparticles can be used without an active agent, but preferably they also include an active agent, which is pro-elastogenic and/or anti-proteolytic. The active agent may be dispersed within the polymeric core and/or attached to the surface of the polymeric core using peptide linkages (e.g., proteolytically cleavable peptide linkages).

Nanoparticles, as the term is used herein, are particles having a size of 1000 nanometers or less. In some embodiments, the particles have a diameter from about 100 nanometers to about 1000 nanometers. In other embodiments, the particles have a diameter from about 200 nanometers to about 500 nanometers. In further embodiments, the particles have a diameter from about 300 to about 400 nanometers, while in yet further embodiments the particles have a diameter from about 325 to about 375 nanometers. The diameter of the nanoparticles refers to their mean hydrodynamic diameter. The hydrodynamic diameter is the measurement that includes the polymeric core along with the functionalization layer and a solvent layer that associates with the nanoparticle. The hydrodynamic diameter can be readily determined using dynamic light scattering (DLS).

The polymer core of the nanoparticles can be formed from one or more natural or synthetic polymers such as, without limitation, polystyrene, polylactic acid, polyketal, butadiene styrene, styreneacrylic-vinyl terpolymer, polymethylmethacrylate, polyethylmethacrylate, polyalkylcyanoacrylate, styrene-maleic anhydride copolymer, polyvinyl acetate, polyvinylpyridine, polydivinylbenzene, polybutyleneterephthalate, acrylonitrile, vinylchloride-acrylates, polycaprolactone, poly(alkyl cyanoacrylates), poly(lactic-co-glycolic acid), and the like. In a preferred embodiment, the polymeric core comprises poly(lactic-co-glycolic acid) (PLGA).

The core of the nanoparticles can be formed from PLGA, which is an FDA-approved polymer, which has been widely used in drug delivery applications, and has been used for DOX encapsulation in previous studies (Misra R, Sahoo S K. “Antibacterial activity of doxycycline-loaded nanoparticles,” Nanomedicine: Infectious Diseases, Immunotherapy, Diagnostics, Antifibrotics, Toxicology and Gene Medicine, Duzgunes N, ed., p. 61-85 (2012), although these DOX-loaded nanoparticles were for anti-bacterial applications. Chitosan can also be used as a carrier for DOX. Cover et al., Int J Nanomedicine., 7:2411-9 (2012).

The nanoparticles generally include an active agent that is pro-elastogenic and/or anti-proteolytic. In some embodiments, the active agent is dispersed within the core. Preferably the active agent is substantially evenly dispersed throughout the polymeric core. Active agents that are pro-elastogenic refer to the characteristics of agents which stimulate elastin synthesis and matrix deposition, while those that are anti-proteolytic are defined as those that prevent elastolysis and matrix degradation. Any suitable active agent can be used. Several biomolecules have been shown to positively impact elastic matrix synthesis in different tissues, including growth factors, matrix glycosaminoglycans, nucleotides, exogenous enzymes, vitamins, and steroids. Specific examples of active agents include cyclic GMP, fibroblast chondroitin sulfate, insulin-like growth factor 1 (IGF-1), dexamethasone, bleomycin, aldosterone, retinoic acid, lysyl oxidase, HA oligomers, TGF-β1, HGF, and proteinase inhibitors such as matrix metalloproteinase inhibitors. Accordingly, in some embodiments, the active agent can be an anti-proteolytic agent, while in further embodiments the active agent can be a matrix metalloproteinase inhibitor. Matrix metalloproteinase inhibitors can affect a particular matrix metalloproteinase, or they can affect a plurality of matrix metalloproteinases. Examples of matrix metalloproteinases (MMPs) include MMP-2, MMP-9, and MMP-12. In some embodiment of the invention, the matrix metalloproteinase inhibitor is doxycycline.

In another embodiment of this invention, the NPs maybe modified/combined with suitable agents for enhancing their delivery and targeting efficiency, as well as their uptake and retention, tracking and/or diagnostic capabilities. Targeting may be enhanced by agents such as but not limited to antibodies targeting fibrin, platelets/platelet receptors, as well as components of the extracellular matrix, or products thereof. Additionally, the use of agents such as super paramagnetic iron-oxide particles (SPIO micro/nanoparticles) can also serve to enhance the targeting specificity of the NPs developed by the inventors.

The active agent can be linked to the surface of the nanoparticle via a peptide linkage in some embodiments. Preferably, the peptide linkage is a proteolytically-sensitive peptide linkage (e.g., an MMP-sensitive linkage) so that the active agent can be released at the target site by proteolytic enzymes. Peptide linkers can be incorporated on the surface of the nanoparticle either via simple incubation with the peptide (Gullotti et al. Pharm Res., 30(8): 1956-1967 (2013)) which is a physical adsorption based process, or chemically using NHS-EDC chemistry (Cheng et al. Biomaterials 28,869-876 (2007)), with both disclosures being incorporated herein by reference.

In some embodiments, an imaging agent is linked to the surface of the nanoparticle via a peptide linkage (e.g., a proteolytically sensitive peptide linkage). Release of the imaging agent at the tissue site of proteolysis enables real-time semiquantitative assessment of tissue proteolysis (e.g., MMP activity). Examples of imaging agents include fluorescent compounds, radioactive isotopes, and MRI contrast agents. For example, in some embodiments, the imaging agent is a fluorescent molecule for fluorescent imaging. Imaging agents have been well-developed in the field of fluorescent imaging, magnetic resonance imaging, positive emission tomography, or immunoassays and, in general, most any imaging agent useful in such methods can be applied to the present invention. Thus, an imaging agent is any composition detectable by spectroscopic, photochemical, biochemical, immunochemical, electrical, optical or chemical means. Useful imaging agents in the present invention include fluorescent dyes (e.g., fluorescein isothiocyanate, AlexaFluor555, Texas red, rhodamine, and the like), near-infrared emitting fluorophores such as indocyanine green, radiolabels (e.g., ³H, ¹⁴C, ³⁵S, ¹²⁵I, ¹²¹In, ¹¹², ⁹⁹mTc), other imaging agents such as microbubbles (for ultrasound imaging), ¹⁸F, ¹¹C, ¹⁵O, (for Positron emission tomography), ⁹⁹mTC, ¹¹¹In (for single photon emission tomography), and chelated lanthanides such as terbium, gadoliniuum, and europium (e.g., chelated gadolinium) or iron (for magnetic resonance imaging). The choice of imaging agent depends on the sensitivity required, ease of conjugation with the compound, stability requirements, available instrumentation, and disposal provisions.

A peptide linkage can be used to associate an active agent or imaging agent to the nanoparticle. Preferably, the peptide linkage is proteolytically-sensitive to allow cleavage of the peptide linkage at the tissue site of proteolysis. A proteolytically sensitive peptide linkage is one that is rapidly cleaved by a proteolytic enzyme. Proteolytically sensitive peptide linkages are well known to those skilled in the art. In some embodiments, the proteolytically sensitive peptide linkage is sensitive to cleavage by MMP. Examples of MMP-sensitive peptide linkages include GPQGIWGQ (Seliktar et al., J Biomed Mater Res., 68A(4):704-716 (2004); Lutolf et al. Proc Natl Acad Sci USA., 100(9):5413-8 (2003)), GPLGVRC (Gulotti et al. Pharm Res., 30(8): 1956-1967 (2013)), and PVGLIG (Chau et al., Int J Cancer.; 118(6):1519-26 (2006)).

The active agent or imaging agent can be conjugated to nanoparticles bearing the conjugated MMP-sensitive peptides via carbodiimide crosslinking chemistry using, for example, 1-ethyl-3-(-3-dimethylaminopropyl) carbodiimide hydrochloride (EDC) and N-hydroxysuccinimide (NHS), which allow conjugation to free carboxyl groups. A wide variety of bioconjugation methods are known to those skilled in the art, which can be used to allow conjugation to various moieties present on the nanoparticle surface. See Greg T. Hermanson, Bioconjugate Techniques, 3^{rd} edition, Academic Press, 2013. The active agent or imaging agent can be attached to the nanoparticle either before the peptide linkers are associated with the nanoparticle, or after the peptide linkers are associated with the nanoparticle.

The elastogenic nanoparticles can be used to carrying varying amounts of an active agent. In some embodiments of the elastogenic nanoparticles provided herein, the weight/weight (w/w) percent of the active agent in the nanoparticles is about 5%. In some embodiments, the w/w percent of the active agent in the nanoparticles is about 2-10%.

The surface of the polymer core is functionalized with a cationic amphiphilic compound. The organic compounds should be biocompatible and suitable for providing a significant amount of positive charge to the nanoparticle. In some embodiments, the nanoparticles have a surface charge from about +10 mV to about +50 mV. In other embodiments, the nanoparticles have a surface charge from about +20 mV to about +40 mV, while in further embodiments the nanoparticles have a surface charge from about +30 mV to about +35 mV.

Cationic amphiphilic compounds are organic compounds including a positive charge and have an amphiphilic character. Amphiphilic character refers to a compound having a polar water-soluble group attached to a water-insoluble hydrocarbon chain, as is found in surfactant molecules. In some embodiments, the positive charge is provided by a quaternary ammonium group. Examples of cationic amphiphilic compounds include dimethyldioctadecylammonium chloride; dioctadecyldimethylammonium bromide (DODAB), didodecyldimethylammonium bromide (DMAB), dodecylamine hydrochloride (DAH), docecyltrimethyl ammonium bromide (DTAB), linear isoform polyethylenimine (linear PEI), branched low MW polyethylenimine (PEI) (of about <25 kDa), branched Low MW polyethylenimine (PEI) (of about <15 kDa), branched Low MW polyethylenimine (PEI) (of about <10 kDa), branched high MW polyethylenimine (of about >1-25 kDa), poly-L-arginine (average or nominal MW of about 70 kDa), poly-L-arginine (average or nominal MW>about 50 kDa), poly-L-arginine (average or nominal MW of about 5 to about 15 kDa), poly-L-lysine (average or nominal MW of about 28 kDa), poly-L-lysine (average or nominal MW of about 67 kDa), poly histidine, ethylhexadecyldimethylammonium bromide, dodecyltrimethyl ammonium bromide, tetradodecylammonium bromide, dimethylditetradecyl ammonium bromide, tetrabutylammonium iodide, DEAE-dextran hydrochloride, and hexadimethrine bromide. A preferred cationic amphiphilic compound is didodecyldimethyl ammonium bromide (DMAB).

### Role of DMAB in Enhancing Elastic Matrix Synthesis

Cationic amphiphiles such as dodecyl trimethylammonium bromide (DTAB) and dodecylamine hydrochloride (DAH) have been shown to stimulate LOX activity and reduce elastolysis (or elastin degradation) by their hydrophobic binding to elastin molecules, as well as their cationic charge which leads to repulsion of elastase (net positively charged at physiological pH). (Jordan et al., Biochemistry, 13:3497-503 (1974)). Anionic hydrophobic agents had the opposite effect.

Accordingly, it was hoped that hydrophobic dodecyl chains of DMAB incorporated on the surface of NPs would facilitate binding to hydrophobic domains on existing elastic matrix structures to provide a means of targeting active agent delivery, that hydrophobic dodecyl chains of DMAB incorporated on the surface of NPs would facilitate coacervation of tropoelastin molecules to enhance nucleation, essential for fiber formation and localized crosslinking, that the positive surface charge on NPs would mediate recruitment of cell-generated lysyl oxidase (LOX; enzyme which mediate the crosslinking of elastin molecules into a mature matrix/fibers), which is negatively charged at physiological pH to enhance the formation of mature, crosslinked elastic fiber/matrix, and that the pro-elastogenic effects of the active agents released from the NPs would be augmented by these mechanisms.

### Role of DMAB in Inhibiting Matrix Elastolysis by MMPs

Previous studies by other groups have demonstrated the ability of cationic long chain compounds to inhibit MMP production and/or activity. (Tezvergil-Mutluay et al., J Dent Res. 90:535-40 (2011)). Mendis et al. demonstrated the ability of a quaternary ammonium glucosamine derivative to inhibit the expression and activation of MMP-2 and -9 in HT1080 fibrosarcoma tumor cells. (Mendis et al., Bioorg Med Chem Lett. 19:2755-9 (2009)). Tezvergil-Mutluay et al. speculated that cationic quaternary ammonium compounds bind electrostatically to negatively charged glutamic acid residues in the active site of MMPs, and also sterically block the active site from recognizing peptide sequences in matrix proteins, and thereby inhibiting their activity. In another study, it was demonstrated that although MMP-7 interacted with anionic lipid membranes, its activity remained unaffected; however, when it bound to cationic lipid membranes, MMP-7 activity was inhibited drastically, and this was attributed to the orientation of the active site of the enzyme towards the positively charged lipid membrane surface. (Ganguly et al., FEBS Lett., 581:5723-6 (2007)).

Based on the results from the inventors' studies which showed the ability of DMAB-functionalized NPs to attenuate MMP-synthesis and activity, they hypothesized that DMAB and other cationic hydrophobic amphiphiles would provide a variety of beneficial effects when incorporated into NP or other biomaterial surfaces. For example, it was hoped that these molecules would act via a direct mechanism, since it is known that cationic glucosamine derivatives to attenuate MMP-2 and -9 expression at the transcriptional level, while also inhibiting their activities. It was also hoped that providing NPs with a positive surface charge that would inactivate the negatively charged glutamic acid residues, which are present in the active site of MMPs and are essential for their activity, by binding to them via electrostatic interactions. In addition, it was hoped that the long hydrophobic chains would sterically prevent the MMPs from interacting with matrix proteins.

The functionalization of NPs with cationic amphiphiles imparts them with multifunctionality in terms of MMP-inhibition and improved elastogenic outcomes via the following attributes/properties/advantages. First, it provides the NPs with a targeting ability to specific tissue/matrix structures within the tissue (e.g., aortic wall). Second, it provides enhanced uptake and retention of these NPs within GAG containing tissues (e.g., the aortic wall) due to their improved targeting and binding capabilities and their cationic surface charge. Third, it provides the NPs with enhanced elastin/elastic matrix binding, due to interactions with hydrophobic domains in elastin and/or tissue.

Functionalization of the NPs also provides a number of other advantages. The elastogenic NPs show improved elastogenic outcomes via recruitment of lysyl oxidase (LOX), which is negative charged at physiological pH. The cationic amphiphiles also cause repulsion of positively charged elastases (i.e., elastin degrading enzymes) due to the positive surface charge on the NPs. Functionalization also provides inhibition of MMP synthesis and activation, and inactivation of the negatively charged glutamic acid residues, which are essential for enzymatic activity of MMPs, due to electrostatic binding to the positively charged NPs. The cationic long-chain surface-modifiers also provide a steric blockade of the active site of MMPs, which prevent it from interacting with matrix proteins and thereby leading to MMP-inhibition. Functionalization also facilitates localized, controlled, sustained release of biomolecules for providing MMP-inhibition and enhanced elastogenic induction of cells producing elastin.

In some embodiments, the elastogenic nanoparticles further comprise a superparamagnetic iron oxide (SPIO). The SPIO can be incorporated into the nanoparticles during formation of the nanoparticles, e.g., during formation of the nanoparticles using the double-emulsion solvent evaporation method. The SPIO is being incorporated into a nanoparticle should have a much smaller size in the nanoparticle range as well. The SPIO nanoparticles have diameters between about 1 and 100 nanometers. The two main forms of superparamagnetic iron oxide are magnetite (Fe₃O₄) and its oxidized form maghemite (γ-Fe₂O₃), both of which are known to be biocompatible materials. Including SPIO in the elastogeneic nanoparticles allows the elastogenic nanoparticles to be rendered mobile and spatially directed using a magnetic field created using strong permanent magnets, which enables their magnetic guidance to target tissue by moving the source of the magnetic field to a location proximate to the target tissue.

Another aspect of the invention provides a method of stimulating elastogenesis in a subject by administering to the subject a therapeutically effective amount of elastogenic nanoparticles comprising a polymeric core having a surface that is functionalized with a cationic amphiphilic compound. As described herein, it has been shown that elastogenic nanoparticles can be useful for stimulating elastogenesis even without including an active agent. However, in some embodiments, the method of stimulating elastogenesis uses nanoparticles that include an active agent dispersed within the polymeric core of the nanoparticle. In further embodiments, the active agent can be an anti-proteolytic agent (e.g., a matrix metalloproteinase inhibitor), which may also have pro-elastogenic effects, or other agents with pro-elastogenic effects and/or anti-proteolytic characteristics.

A “subject”, as used therein, can be a human or non-human animal. Non-human animals include, for example, livestock and pets, such as ovine, bovine, porcine, canine, feline and murine mammals, as well as reptiles, birds and fish. Preferably, the subject is human.

In some embodiments, the elastogenic nanoparticles can be administered to a subject who is in need of treatment to stimulate elastogenesis based on the diagnosis of a particular matrix disruptive disease, injury process, or disorder in that subject. For example, in some embodiments, the subject has been diagnosed as having an abdominal aortic aneurysm (AAA). This approach seeks to concurrently address/solve two critical factors which play a role in AAA evolution and progression: (i) proteolytic matrix degradation (particularly elastin/elastic matrix) by MMPs, and (ii) inherently poor regeneration/repair of disrupted/lost elastin and elastic matrix by adult vascular SMCs and SMCs of an activated/diseased phenotype, such as aneurysmal SMCs.

Experiments conducted by the inventors showed that surface functionalization of the NPs with cationic long-chain compounds such as DMAB, DTAB and DAH was responsible for enhanced elastogenic outcomes in vitro in aneurysmal SMC cultures as well as in elastin binding studies, while concurrently causing MMP-2 inhibition. Additionally, it was shown that the localized, controlled, sustained DOX release also was effective in inhibiting MMP-2 synthesis and activity. More importantly, considering the limitations associated with open AAA repair, which include (i) age and (ii) patient morbidity and mortality, as well as those with endovascular AAA repair, which include (i) endoleaks and (ii) graft migration, use of elastogenic nanoparticles represents a non-surgical modality for AAA therapy.

The elastogenic NPs offer promise in a variety of applications in regenerative medicine. In one embodiment, the NPs can be combined with stent grafts (endovascular AAA therapy) to develop a multi-faceted approach to slow and/or arrest AAA growth. It has been shown that although stent grafting within AAAs excludes the AAA wall from chronic hypertensive hemodynamic forces, proteolytic matrix disruption continues in the AAA wall to result in continued AAA wall weakening to rupture. DOX-NP therapy can attenuate this process. There is also potential to modify the stent grafts with these NPs to provide controlled release of DOX towards (elastic) matrix stabilization and regenerative repair. In another embodiment, the NPs can be integrated with periadventitial scaffolds or patches or foams or gels to release therapeutics from the outside of the vessel. Progress in engineering/regenerating tissue-type specific elastic matrices for vascular, pulmonary, pelvic floor, vocal fold tissue, skin, and bladder tissue has been described by the inventors. See Sivaraman et al., Drug Deliv Transl Res. 2(5):323-350 (2012), the disclosure of which is incorporated herein by reference.

In another embodiment, DOX-loaded NPs functionalized with cationic amphiphiles can be used within bandages or patches for skin repair & wound healing applications. Skin contains 2-4% elastin, and is an important component of skin, as its regeneration is essential in recovery from burn or chronic wounds. DOX has been used topically for wound healing. (Stechmiller et al., Biol Res Nurs., 11:336-44 (2010)).

The elastogenic NPs may also be utilized in skin/beauty creams, for their enhanced permeation into the epidermal/dermal layer, as well as functional matrix regenerative outcomes within towards keeping skin and its matrix components healthy. This could have functional effects in treating elastosis, i.e., changes in the strength and elasticity of skin.

In another embodiment, the elastogenic nanoparticles could be used for periodontal therapy in subjects diagnosed as having periodontal disease. Elastogenesis can be important for stimulating growth of gum tissue, and in some embodiments controlled DOX release may be useful in dental/periodontal therapy, on account of its antibacterial properties (Tonetti et al., J Clin Periodontol., 39:475-82 (2012)), as well as its MMP-inhibitory properties to counter periodontal inflammation (Lee et al., J. Periodontol. 75: 453-463 (2004)).

In a further embodiment, the elastogenic NPs prepared in an aerosol form could be used as vehicles for pulmonary drug delivery as well as in enhancing pulmonary elastogenic outcomes and inhibiting MMPs within pulmonary tissue. Elastin/elastic fibers mediate the extension and recoil of blood vessels, airways and alveoli in the lungs, accounting for ˜30% of total lung tissue (on a dry weight basis). (Moore et al., J Voice, 26:269-75 (2011)). The elastic fibers and matrix degradation/damage occurs in acute and chronic lung diseases, and are coupled with MMP-2, -9 and -12 upregulation. Greenlee et al., Physiol Rev., 87:69-98 (2007). This can lead to reduction in pulmonary function (Crouch E., Am J Physiol., 259:L159-L84 (1990)), and although the damaged elastic fibers can be repaired to some extent, the reconstruction of the highly complex alveolar structure is difficult.

In a yet further embodiment, elastogenic NPs can be used as vehicles for drug delivery to the pelvic floor tissue, as well as in enhancing elastogenic outcomes and inhibiting MMPs within pelvic floor tissue. The structural properties of the connective tissue of the pelvic floor are mediated by elastin and collagen present within the endopelvic fascia. Unlike SMCs in vascular and other connective tissue, healthy SMCs in the pelvic tissue continue to produce elastin and elastic matrix well into adulthood. (Rahn et al., Am J Physiol Regul Integr Comp Physiol., 295:R1351-R8 (2008)). Studies have demonstrated enhanced elastin fiber production and crosslinking in the vaginal wall postpartum, on account of its distension during the delivery process. However, this is also accompanied by increased elastin remodeling by MMP-2 and MMP-9. Drewes et al., Am J Pathol, 170:578-89 (2007). This leads to weakening of the pelvic tissue and the development of a condition called pelvic organ prolapse (POP) in some individuals. Genetic impairment of the elastic fiber assembly machinery also appears to be linked to the susceptibility to develop POP.

The cervicovaginal mucus is a barrier to NP delivery, with 200 nm NPs unable to diffuse through this layer as they are larger than the pore size of this mucus network mesh. (Lai et al., Proc Natl Acad Sci USA, 104:1482-7 (2007)). However, functionalization of these NPs with PEG imparts them with a neutral charge, and enhanced their transport through the cervicovaginal layer. (Lai et al., Adv Drug Deliv Rev., 61:158-71 (2009)). The DTAB and DAH-functionalized NPs prepared by the inventors have potential applications in this scenario on account of their near neutral yet net positive charge, with their MMP-inhibitory properties enhancing outcomes, along with their improved elastogenic benefits.

Polymeric meshes or slings are often used in umbilical/abdominal hernia repair and even POP to support internal organs or vagina from extending or descending. In another embodiment, NPs incorporating MMP-inhibitory (e.g., DOX) or elastogenic biomolecules (e.g., HA-o & TGF-β1) may be integrated with these meshes to provide an active tissue regenerative or anti-proteolytic stimulus to affect active tissue repair/healing. See Sylvester et al., Acta Biomater. 9(12):9292-302 (2013) and Venkataraman et al., J Tissue Eng Regen Med. April 16. doi: 10.1002/term.1889 (2014), which show that TGF-β and HA-o can be released from PLGA using NPs that were surface functionalized with PVA, which imparts negative surface charge.

Oral DOX dosing has been shown to improve healing following rotator cuff repair, due to its ability to inhibit MMPs. Accordingly, in a further embodiment, the multifunctional DOX-loaded NPs can be incorporated within patches/scaffolds used in rotator cuff repair, for localized, controlled, long-term DOX delivery, along with concurrent inhibition of MMPs and improved elastogenic outcomes towards healing.

The NPs can also be embedded within biomaterial scaffolds for multiple multi-purpose applications, such as their incorporation within tissue engineered constructs to overcome the diffusional limits for nutrients and other biomolecules, towards reduced spatial and temporal heterogeneity in cell fate and extracellular matrix (ECM) regeneration.

### Formulation and Administration

Another aspect of the invention provides a pharmaceutical formulation including elastogenic nanoparticles and a pharmaceutically acceptable carrier, wherein the elastogenic nanoparticles comprise a polymeric core having a surface that is functionalized with a cationic amphiphilic compound, and an active agent which has pro-elastogenic properties and/or anti-proteolytic effects (e.g., a matrix metalloproteinase inhibitor such as doxycycline) dispersed within the core. Examples of pharmaceutical formulations include topical formulations and parenteral formulations.

The elastogenic nanoparticles of the invention are administered to a subject to stimulate some aspect of elastogenesis, such as elastin precursor synthesis, or precursor crosslinking and elastic fiber assembly. In some embodiments, the elastogenic nanoparticles are administered intravenously. However, in other embodiments, the elastogenic nanoparticle may also be administered topically, orally, intramuscularly, intradermally, intraperitoneally, intralymphaticly, percutaneously, or by scarification, subcutaneous injection or other parenteral routes.

In each of the above embodiments, a nanoparticle of the invention maybe combined with a pharmaceutically acceptable vehicle or carrier to provide a pharmaceutical composition. The elastogenic nanoparticles may be present in a pharmaceutical composition in an amount from 0.001 to 99.9 wt %, more preferably from about 0.01 to 99 wt %, and even more preferably from 0.1 to 95 wt %. For instance, in embodiments where these elastogenic nanoparticles are administration by injection (e.g., intraperitoneally, intravenously, subcutaneously, intramuscularly, etc.), the compositions are preferably combined with pharmaceutically acceptable vehicles such as saline, Ringer's solution, dextrose solution, and the like.

The compositions for administration will commonly comprise a suspension of the elastogenic nanoparticles in a pharmaceutically acceptable carrier that is selected so as not to affect the biological activity of the combination. Examples of aqueous carriers are distilled water, physiological phosphate-buffered saline, Ringer's solutions, dextrose solution, and Hank's solution. These suspensions are sterile and generally free of undesirable matter. These compositions may be sterilized by conventional, well-known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

In some embodiments, the elastogenic nanoparticles are provided in topical formulations, such as liquid or solid oil-in-water emulsions, water-in-oil emulsions, multiple emulsions, microemulsions, PET-emulsions, bickering emulsions, hydrogels, alcoholic gels, lipogels, one or multiphase solutions, foams, ointments, plasters, suspensions, shampoos, powders, cremes, cleanser, soaps and other usual compositions. The topical formulations can also include pharmaceutical adjuvants and additives, such as preservatives/antioxidants, fatty substances/oils, water, organic solvents, silicones, thickeners, softeners, emulsifiers, sunscreens, antifoaming agents, moisturizers, fragrances, surfactants, fillers, sequestering agents, anionic, cationic, nonionic or amphoteric polymers or mixtures thereof, propellants, acidifying or basifying agents, dyes, colorants, pigments, and the like.

Single or multiple administrations of the compositions may be administered depending on the dosage and frequency as required and tolerated by the subject. In any event, the administration regime should provide a sufficient quantity of the composition of this invention to effectively treat the subject. The formulated elastogenic nanoparticles can be administered as a single dose or in multiple doses.

One of skill in the art will recognize that the amount of the elastogenic nanoparticles in these formulations can vary widely, and will be selected primarily based on fluid volumes, viscosities, body weight and the like in accordance with the particular mode of administration selected and the subject's needs. In one embodiment, the amount of elastogenic nanoparticle administered is between about 0.25 μmol/kg and about 3 μmol/kg. In another embodiment, the amount of elastogenic nanoparticle administered is between about 0.5 μmol/kg and about 1.5 μmol/kg. In yet another embodiment, the amount of elastogenic nanoparticle administered is about 1 μmol/kg. In still another embodiment, the amount of nanoparticle administered is between about 0.3 g/kg and about 0.4 g/kg.

In further embodiments, the pharmaceutical formulation coats the surface of, or is admixed within, a biocompatible scaffold. Scaffolds are biocompatible materials shaped to provide a support for tissue engineering, and can be prepared from a variety of natural or synthetic materials. The elastogenic nanoparticles of the invention may be included in or on a scaffold in order to stimulate elastogenesis to facilitate tissue constructions. When including elastogenic nanoparticles on the surface of the scaffold, it is preferable to include the nanoparticles in a biodegradable polymer such as a poly-orthoester or poly-anhydride that will initially retain the elastogenic nanoparticles, but will gradually release the nanoparticles at the tissue engineering site as the polymer degrades.

The following examples are included for purposes of illustration and are not intended to limit the scope of the invention.

### EXAMPLES

### Example 1

### Multifunctional Nanoparticles for Doxycycline Delivery Towards Localized Elastic Matrix Stabilization and Regenerative Repair

### Materials and Methods

Isolation and Culture of SMCs from Elastase Perfusion-Induced Rat AAAs

All animal procedures were conducted with the approval of the Institutional Animal Care and Use Committee at the Cleveland Clinic (ARC #2010-0299). The clinic's animal facility is AAALAC approved and has animal assurance (#A3145-01). Aneurysmal rat aortic SMCs (EaRASMCs) were isolated from adult male Sprague-Dawley rats (n=3) at 14 days post-AAA induction via elastase infusion. The aortae were cut open longitudinally and the intimal layer scraped off gently with a scalpel. The medial layer was then dissected from the underlying adventitial layer, chopped into ˜0.5 mm long slices and washed twice with warm, sterile phosphate-buffered saline (PBS). These were pooled and enzymatically digested in DMEM-F12 cell culture medium (Invitrogen, Carlsbad, Calif.) containing 125 U mg⁻¹ collagenase (Worthington Biochemicals, Lakewood, N.J.) and 3 U mg⁻¹ elastase (Worthington Biochemicals) for 30 min at 37° C., centrifuged (400 g, 5 min) and cultured for over 2 weeks in T-75 flasks. The cells were cultured in DMEM-F12 medium (Invitrogen) supplemented with 10 vol. % fetal bovine serum (FBS; PAA Laboratories, Etobicoke, Ontario) and 1 vol. % penicillin-streptomycin (PenStrep; Thermo Fisher, South Logan, Utah). The primary EaRASMCs obtained from these tissue explants were propagated over 2 weeks, and passaged when they attained confluence.

Primary healthy rat aortic smooth muscle cells (RASMCs) were similarly isolated from aortae excised from healthy adult Sprague-Dawley rats (n=3), and propagated for cell culture experiments as described. Kothapalli et al., Acta Biomater 6:170-8 (2010).

### Determination of Appropriate PLGA NP Size for Delivery to SMC Cultures

An acceptable PLGA NP size based on their relative exclusion by cells in the extracellular space was determined by incubating fluorescent PLGA NPs (FITC-PLGA; Fluorophorex™, Phosphorex, Inc., Fall River, Mass.) of three different sizes (100, 200 and 500 nm hydrodynamic diameter) with cultured EaRASMCs. Briefly, the EaRASMCs were seeded at 1.5×10⁴ cells well⁻¹ in sterile, two-well Permanox chamber slides (Nalge Nunc International, Rochester, N.Y.) and cultured in DMEM-F12 cell culture medium supplemented with 2 vol. % FBS and 1 vol. % PenStrep. The FITC-PLGA NPs were added to the EaRASMCs at a concentration of 0.2 mg ml⁻¹ and incubated at 37° C. for 48 h. The cells were then fixed with 4 vol. % formaldehyde, labeled with the lipophilic membrane stain DiI (Invitrogen, Carlsbad, Calif.), and then mounted with Vectashield containing the nuclear dye 4′,6-diamidino-2-phenyindole (DAPI; Vector Laboratories, Burlingame, Calif.). Imaging to visualize NP uptake or exclusion was performed using a spectral laser scanning confocal microscope (Leica TCS SP2, Leica Microsystems, Inc., Buffalo Grove, Ill.). To visualize and confirm the intracellular presence or extracellular exclusion of NPs, z-stack maximum projections (overlays) were created from images acquired at 1 μm intervals across the thickness of the cell layers using Leica LAS AF software. Outcomes were deduced from a minimum of 10-12 images per NP size.

### Formulation of DOX-Loaded PLGA NPs

Poly(DL-lactic-co-glycolic acid) nanoparticles (PLGA; 50:50 lactide:glycolide; inherent viscosity 0.95-1.20 dl g⁻¹ in hexafluoroisopropanol; Durect Corporation, Birmingham, Ala.) loaded with doxycycline hyclate (DOX; Sigma-Aldrich, St. Louis, Mo.) were prepared by a double emulsion solvent evaporation technique. Song et al., J Control Release, 43:197-212 (1997). Briefly, PLGA was dissolved in chloroform (Fisher Scientific, Fair Lawn, N.J.) at a concentration of 2.5-3.0% w/v. An aqueous DOX solution at three different loadings (2, 5 and 10 wt. % ratios of DOX:PLGA) was emulsified into the PLGA solution using a probe sonicator (Q500; QSonica LLC, Newtown, Conn.) for 30 s on ice, at an amplitude setting of 20%. The water-in-oil emulsion thus formed, was further emulsified into an aqueous solution of 1% w/v didodecyldimethylammonium bromide (DMAB; Sigma-Aldrich) using the probe sonicator for 30 s on ice at 20% amplitude setting, to form a water-in-oil-in-water emulsion. This second emulsion was stirred for 16 h at room temperature and then desiccated for 1 h under a vacuum to remove any residual chloroform. The NPs formed were recovered by ultracentrifugation at 35,000 rpm (Beckman L-80, Beckman Instruments, Inc., Palo Alto, Calif.). The NPs were washed twice with nanopure water to remove residual DMAB and unencapsulated DOX and then lyophilized for 48 h to obtain a dry powder. Suitable precautions were taken to ensure minimal exposure to light during the formulation and release process, as tetracyclines are known to be light sensitive.

Efficiency of DOX Encapsulation within Nanoparticles

The supernatants from the washing/ultracentrifugation steps were pooled for each individual NP formulation. The unencapsulated DOX was quantified via high-performance liquid chromatography (Waters Separation Module 2690 HPLC, Waters Corporation, Milford, Mass.) coupled with a triple quadrupole mass spectrometer (Micromass Quattro Ultima; Waters Corporation). The total amount of DOX encapsulated in the NPs and the overall encapsulation efficiencies were determined by subtracting the total amount of unencapsulated DOX from the total weighed amounts of DOX added during NP formulation.

### Size and Surface Charge Measurements on DOX-Loaded PLGA Nanoparticles

Mean hydrodynamic diameters of the NPs were determined using a dynamic light scattering technique, and the mean zeta potentials (i.e. surface charge) of these NPs were determined via a phase analysis light scattering technique using a commercial particle-sizing system (PSS/NICOMP 380/ZLS, Particle Sizing Systems, Santa Barbara, Calif.). Peetla C, Labhasetwar V., Langmuir, 25:2369-77 (2009).

### Determining Cytotoxicity of DOX-Loaded PLGA NPs

Cytotoxicity of PLGA NPs was assessed using a LIVE/DEAD viability assay (Invitrogen, Inc.) applied to EaRASMCs that were incubated with the NPs. Briefly, the EaRASMCs were seeded at 1.5×10⁴ cells well⁻¹ in sterile, two-well Permanox chamber slides (Nalge Nunc International) and allowed to adhere over a 48 h period in DMEM-F12 cell culture medium supplemented with 2 vol. % FBS and 1 vol. % PenStrep. The DOX-PLGA NPs (2%, 5%, 10% DOX) were added to the EaRASMCs at concentrations of 0.1, 0.2 and 0.5 mg ml⁻¹ and incubated for 48 h prior to assessing their viability. PLGA NPs containing no DOX were tested as a control. Stained cells were viewed using an Olympus IX51 fluorescence microscope (Olympus America, Center Valley, Pa.). Six different regions were assessed for each replicate culture.

Characterizing DOX Release from PLGA NPs In Vitro

DOX release from NPs was carried out in phosphate-buffered saline (PBS, pH 7.4; Sigma-Aldrich) at 37° C. on a shaker at 100 rpm. Briefly, 1.5 ml polypropylene microcentrifuge tubes (n=3 per formulation) were filled with 1.0 ml of NP suspensions containing 0.2, 0.5 and 1.0 mg ml⁻¹ NPs, respectively. Release in each case was carried out over 40 days. At each analysis time point, the samples were centrifuged (14,000 rpm, 30 min, 4° C.) in a microcentrifuge (Beckman Microfuge 16®, Beckman Coulter, Inc.), the supernatants were withdrawn to quantify the DOX content and the volume was replenished with fresh PBS. The DOX released was quantified by ultraviolet spectrophotometry (SpectraMax M2, Molecular Devices, Inc., Sunnyvale, Calif.). DOX absorbance at 270 nm was calibrated to its concentration using serial dilutions of a 1.0 mg ml⁻¹ DOX solution. The DOX standards were incubated under the same conditions as the NP samples to avoid any time and temperature-dependent degradation of DOX.

### Experimental Design for Cell Culture

For cell culture experiments intended to investigate the impact of DOX-PLGA NPs on cellular matrix synthesis, EaRASMCs (passages 2-5) were seeded at 3×10⁴ cells well⁻¹ and cultured over 21 days in six-well plates (A=10 cm²; BD-Biosciences, Franklin Lakes, N.J.). The cells were cultured in DMEM-F12 supplemented with 2 vol. % FBS, 1 vol. % PenStrep and 100 ng m⁻¹ TNF-α (Pepro-Tech, Inc, Rocky Hill, N.J.) to simulate an aneurysmal or activated microenvironment in culture. NPs loaded with 2 wt. % DOX were cultured with cell layers at concentrations of 0.2, 0.5 and 1.0 mg ml⁻¹ NPs (n=6 per concentration) for 21 days. EaRASMCs cultured with DOX-unencapsulated NPs (0.5 mg ml⁻¹ NPs) served as the active-agent (DOX) control. Healthy, passage-matched RASMCs were similarly seeded, without any NPs, as the cell-type controls. Fresh medium was added to the cultures weekly, with the spent medium slowly pipetted out from individual wells, pooled for each time point and stored at -20° C. The NPs were almost all bound to the cell layer, and were not removed along with the spent medium. The pooled spent medium aliquots were biochemically assayed along with their corresponding cell layers, which were harvested after the 21 days of culture.

For Western blots and zymography to evaluate the effect of DOX released from the NPs on the proteolytic response, EaRASMCs were seeded at 2×10⁵ cells well⁻¹ in a six-well plate and cultured for 7 days in DMEM-F12 supplemented with 5 vol. % FBS and 1% PenStrep, along with 100 ng ml⁻¹ TNF-α. PLGA NPs (0 wt. % DOX active-agent control, 2, 5 and 10 wt. % DOX loading at 0.2 mg ml⁻¹ of NPs) were added to the EaRASMCs at day 1 after seeding (n=6 per treatment).

### DNA Assay for Cell Proliferation

The DNA content of the cell layers was measured via a fluorometric assay to determine the effects of DOX released from the NPs on RASMC and EaRASMC proliferation. The cell layers were harvested on days 1 and 21 of culture in NaCl-Pi buffer, sonicated on ice and assayed for DNA content. The cell density was calculated assuming 6 pg DNA cell⁻¹. Labarca C, Paigen K., Anal Biochem, 102:344-52 (1980).

### Fastin Assay for Elastin

The total elastin content in the cell layer (alkali-soluble and insoluble fractions) and the tropoelastin (soluble elastin precursor) released in the cell culture medium were quantified using a Fastin assay (Accurate Scientific and Chemical, Westbury, N.Y.). The cell layers were harvested after 21 days of culture, resuspended in NaCl-Pi buffer and sonicated on ice to homogenize the cell layer. This suspension thus obtained was digested with 0.1 N NaOH (1 h, 98° C.), then centrifuged to yield a pellet containing mature, highly cross-linked alkali-insoluble elastin, with the supernatant fraction containing less cross-linked alkali-soluble elastin. The alkali-insoluble elastin was then converted into a soluble form prior to quantification, as the Fastin assay can only quantify soluble α-elastin. The pellets obtained after the NaOH digestion step were dried and solubilized with 0.25 M oxalic acid (1 h, 95° C.), after which they were pooled and centrifuge-filtered (3000 rpm, 10 min) in microcentrifuge tubes (Amicon® Ultra, 10 kDa molecular weight cut-off; Millipore, Inc., Billerica, Mass.). These soluble and insoluble matrix elastin fractions, as well as the tropoelastin fraction in the cell culture medium, were then measured using the Fastin assay. The amounts of elastin measured were also normalized to their corresponding DNA amounts so as to provide an accurate comparison between the different treatments.

### Western Blots for MMP-2 and -9 Protein Expression

MMP-2 and -9 production by activated EaRASMCs in the absence and presence of the PLGA NPs and NP-released DOX was semi-quantitatively assessed using Western blots. After 7 days of culture, as described in Section 2.8, the cell layers were harvested in RIPA buffer (Thermo Scientific) containing Halt™ protease inhibitor (Thermo Scientific), and assayed for total protein content using a bicinchonic acid assay kit (Thermo Scientific). Maximum volumes of sample protein (15.6 μl) were then loaded under reduced conditions into each lane of a 10% sodium dodecylsulfate (SDS)-polyacrylamide gel electrophoresis gel (Invitrogen), along with a BenchMark™ pre-stained molecular weight ladder (Invitrogen). The gels were then transferred dry onto nitrocellulose membranes (iBlot® Western Blotting System, Invitrogen). The membranes were blocked with the Odyssey Blocking Buffer (LI-COR Biosciences, Lincoln, Nebr.) for 1 h, following which they were immunolabeled for 16 h at 4° C. with a rabbit polyclonal antibody against MMP-2 (1:500 dilution; Abcam, Cambridge, Mass.) and a rabbit monoclonal antibody against MMP-9 (1:500 dilution; Millipore, Inc.), with a mouse monoclonal antibody against b-actin (1:1000 dilution; Sigma-Aldrich) as the loading control. Secondary antibody labeling was carried out for 1 h at room temperature using IRDye® 680LT goat-anti-rabbit (1:15,000 dilution) and IRDye® 800CW goat antimouse (1:20,000 dilution) polyclonal antibodies (LI-COR Biosciences). Fluoro-luminescent detection of the protein bands was then carried out using a LI-COR Odyssey laser-based scanning system. The intensities of active MMP-2 bands on all gels were quantified using ImageJ software, expressed in terms of relative density units (RDU) and normalized to the intensity of their respective β-actin bands to enable comparison between the different test cases within the same blot. The normalized band intensities of active MMP-2 for the PLGA NP-treated and other test cultures were further normalized to those of the NP-untreated control cultures to determine the fold change(s) in production of active MMP-2 and the statistical significance of the differences between them. The results presented in this manuscript were averaged from five replicate gels run per culture treatment.

### Gel Zymography for MMP-2 and MMP-9 Activity

Treatment-specific effects on cellular MMP-2 and -9 activities were analyzed via gel zymography. Briefly, volumes of the cell layer samples (harvested in RIPA buffer containing protease inhibitor) equivalent to 10 μg of protein were loaded into each lane of a 10% zymogram gel (Invitrogen), along with a BenchMark™ pre-stained molecular weight ladder (Invitrogen). The gel was run for 2 h at 125 V. The gels were then washed in a buffer containing 2.5 vol. % Triton X-100 for 30 min to remove SDS and incubated overnight in a substrate/development buffer to activate the MMPs. The gels were stained with Coomassie brilliant blue solution for 45 min and destained for 90 min, when clear bands appeared visible against the blue background of the gel. The band intensities (RDU) of the bands obtained for test cultures were measured using ImageJ software and normalized to those obtained for the NP-untreated control cultures to determine fold changes in MMP-2 activity. Data was acquired from three independent replicate gels.

### Immunofluorescence Detection of Elastic Matrix Proteins

For immunofluorescence studies, EaRASMCs were seeded in sterile, two-well Permanox® chamber slides (Nalge Nunc International) at a cell density of 5×10³ cells well⁻¹ in DMEM-F12 cell culture medium supplemented with 5 vol. % FBS and 1 vol. % Pen-Strep. DOX-loaded PLGA NPs (2 and 10 wt. % to PLGA; 0.2 mg ml-1 NP concentration) were added to the cultures 1 day later, with 0% DOX-PLGA NPs added as the active-agent control. EaRASMCs cultured without any NPs served as the treatment control. After 21 days of culture, the cell layers were fixed with 4% w/v paraformaldehyde and treated with rabbit anti-rat polyclonal antibodies against elastin (Millipore) and LOX (Abcam), both at 1:100 v/v dilutions. The detected proteins were visualized with Alexa633-conjugated IgG secondary antibodies (1:1000 v/v dilution; Invitrogen). The spatial extent of the cell bodies was defined by labeling filamentous actin using Alex Fluor 488 Phalloidin (1:50 dilution; Invitrogen). The cell layers were mounted with Vectashield containing DAPI, which labels the cell nuclei. A minimum of six regions per cell layer from a total of n=3 replicate treatments were imaged to assess representative outcomes. Imaging was carried out by confocal microscopy (Leica TCS SP5 II, Leica Microsystems, Inc.). To visualize and confirm the intracellular or extracellular localization of elastin and LOX, z-stack maximum projections (overlays) were created from images acquired at 0.5 μm intervals across the thickness of the cell layers using Leica LAS AF software.

Impact of Surface Modification of NPs on their Binding Affinity to Elastin

PLGA NPs were formulated using a protocol similar to that described earlier, with 1% w/v poly(vinyl) alcohol (PVA; Sigma-Aldrich) as the surfactant. The NPs were lyophilized, resuspended in nanopure water and then functionalized with 1% w/v of dodecyltrimethyl ammonium bromide (DTAB; Sigma-Aldrich) or dodecylamine hydrochloride (DAH; Acros Organics, Fair Lawn, N.J.) by sonication for 30 s on ice. Labhasetwar et al., J Pharm Sci, 87:1229-34 (1998). This alternative method for surface modification of PLGA NPs was necessary as DTAB and DAH did not form nanoparticles at the 1% w/v concentration previously used with DMAB, reasons for which are described in the Results section. Surface-modified PLGA NPs were ultracentrifuged to remove residual traces of stabilizer, then lyophilized. The size and charge of these NPs prepared via surface functionalization with DTAB and DAH, as well as those prepared using 1% w/v PVA alone, were determined as described earlier. To enable their fluorometric detection, PLGA NPs were formulated with the three different cationic amphiphile surface modifiers (DMAB, DTAB and DAH), with 1.0 mg of BSA-Cy5 (NanoCS, New York, N.Y.) encapsulated within. Negatively charged PLGA NPs prepared using PVA as the sole surfactant were also tested as controls.

To compare the elastin binding capacities of the different surface-modified PLGA NPs, bovine aortic elastin strips (Elastin Products Company, Owensville, Mo.) were hydrated in PBS, punched into 4.5 mm diameter pieces and placed in 180 μl PBS in individual wells of a 96-well plate. Aliquots of each of the surface-modified NPs (20 μl) were added to each well at three different NP concentrations (0.1, 0.2 and 0.5 mg of NPs; n=3 per NP type) and incubated with the elastin strips for 30 min. To assess non-specific binding of NPs to the wells (if any), empty wells were also incubated with NPs at the three concentrations mentioned. At the end of the incubation step, the supernatant solution was collected and all the wells (elastin and empty blanks) were washed four times with PBS, the wash solutions then being collected and added to the supernatant for fluorometric analysis for NP content. The dilution of the supernatant due to the addition of solution from the four wash steps was taken into account in the subsequent quantification of NP binding. NP binding to elastin strips and empty wells was quantified from the fluorescence of the supernatant a nm, (λ_{excitation}=650 nm, λₑₘᵢₛₛᵢₒₙ=690 nm) based on a calibration curve prepared from serial dilutions of suspensions containing known concentrations of the different NPs. The total NP adherence to the elastin was obtained by subtracting the amount of NPs adherent to the empty wells from the amount adherent in the wells containing the elastin strip, and is expressed as a percentage of the amount added.

### Effect of Surface-Modified PLGA NPs on Cellular LOX Activity

EaRASMCs were seeded in six-well plates at a seeding density of 6×10⁴ cells per well, and cultured with DMEM-F12 supplemented with 5 vol. % FBS and 1 vol. % PenStrep. PLGA NPs surface modified with DMAB, DTAB, DAH and PVA, formulated as described in earlier sections, were then added to the cell layers at a nanoparticle concentration of 0.2 mg ml⁻¹ of NPs. The cells were cultured with the NPs for 21 days. The cell culture medium was changed every week and the spent cell culture medium from each individual well was collected, pooled in individual tubes and frozen at 20° C. The cell layer was harvested at the end of the culture in RIPA buffer, and assayed for LOX activity along with the pooled spent culture medium.

Hydrogen peroxide released upon deamination of diamino compounds by LOX was detected using a fluorometric Amplex® Red assay (Invitrogen), following a modified protocol. Briefly, 50 μl of samples from cell culture medium or cell layers was mixed with 50 μl of a 50 mM sodium phosphate reaction buffer (pH 7.4) containing 1.2 M urea, 10 mM 1,5-diaminopentane (Sigma-Aldrich), 0.2 U ml⁻¹ horseradish peroxidase (HRP; Invitrogen) and 10 μM Amplex® Red (Invitrogen). Control samples (50 ml) were mixed with sodium phosphate reaction buffer containing only 0.2 U ml⁻¹ HRP and 10 μM Amplex® Red. A calibration curve was constructed based on the absorbance of serial dilutions (0-10 μM) of hydrogen peroxide standards (Invitrogen) treated with the sodium phosphate buffer containing 0.2 U ml⁻¹ HRP and 10 μM Amplex® Red, measured at 560 nm. The absorbance of the control samples was subtracted from that of the individual test case samples to obtain a measure of the hydrogen peroxide released in response to the deamination of urea and 1,5-diaminopentane by the LOX in the medium and cell layer samples.

### Isolating the Effects of Cationic NP Surface Modifiers on MMP-2 Production and Activity

The cell layers isolated (in RIPA buffer) for the LOX activity assay from EaRASMC cultures treated with DMAB, DTAB, DAH and PVA NPs, along with untreated controls were also examined for MMP-2 expression (western blots) and activity (gel zymography), as described earlier. The intensity of the active MMP-2 bands in western blots were normalized to the intensity of their corresponding β-actin bands and compared to those of NP-untreated control cultures to determine the fold-change in MMP-2 expression. For gel zymography, all lanes were loaded with sample volumes equivalent to 12 μg protein, and the intensities of the MMP-2 bands from zymograms were directly normalized to that of the NP-untreated controls to ascertain fold-change in MMP-2 activity for the NP-treated cultures. All data was acquired from 3 independent replicate gels.

### Statistical Analysis

All experimental data presented (n=6/condition, unless stated otherwise) are mean values with standard deviation (SD). Statistical significance of differences between mean values for different samples and conditions was evaluated using a Student's t-test, with p<0.05 considered as statistically significant.

### Results

### Determination of Optimal PLGA NP Size for Delivery to SMC Cultures

The intracellular uptake of FITC-PLGA NPs of different sizes was examined to determine the NP size or size-range which would preferably not be internalized by the SMCs, but instead would predominantly remain in the extracellular space for effecting functional improvements to cell-mediated elastic matrix assembly, which occurs extracellularly. Confocal micrographs of EaRASMC cultures incubated with FITC-PLGA of 100, 200 and 500 nm were obtained. The FITC-PLGA NPs of 100 nm and 200 nm sizes were internalized by the EaRASMCs, while NPs of 500 nm size were excluded in the extracellular space. Thus, it was concluded that the PLGA NPs should be of sizes >200 nm to enable them to be excluded by cells in the extracellular space.

### Formulation and Characterization of DOX-Loaded PLGA NPs

The physical characteristics of PLGA NPs, such as size and zeta potential, are typically controlled by the formulation method(s), with the single or double emulsion solvent evaporation methods being the most common method used for PLGA NP formulation. DOX-free PLGA NPs formulated via a single emulsion solvent evaporation technique using 1% w/v DMAB as the stabilizer, exhibited a mean hydrodynamic diameter (or hydrated particle size) of 332.7±8.3 nm, and a ζ-potential of +33.8±0.4 mV (shown in Table 1). As DOX is a water-soluble drug, we utilized a double emulsion solvent evaporation technique for its encapsulation within the PLGA NPs. DOX encapsulation at 2, 5, and 10% (w/w ratios to PLGA) did not drastically affect the size or surface charge of these PLGA NPs, as indicated in Table 1. Overall, these NPs exhibited a relatively uniform size distribution and surface charge, as indicated in Table 1. The overall encapsulation efficiency of DOX in the PLGA NPs was in the range of 39-42%.

TABLE 1
Size and surface charge of DOX-encapsulated PLGA NPs. (n = 6
replicate formulations, mean ± 95% CI).

| | DOX loading [%; w/w ratio to PLGA] | | | |
|---|---|---|---|---|
| | 0 | 2 | 5 | 10 |
| Size [nm] | 332.7 ± 8.3 | 357.9 ± 79.1 | 379.4 ± 14.4 | 274.0 ± 6.7 |
| ζ-potential | 33.8 ± 0.4 | 34.2 ± 6.6 | 25.3 ± 2.5 | 31.1 ± 3.7 |
| [mV] | | | | |
| DOX | N/A | 40.1 ± 5.7 | 42.3 ± 7.4 | 38.7 ± 7.4 |
| encapsulation | | | | |
| [%] | | | | |

### In Vitro Determination of Concentration-Specific Cytotoxicity of DOX-Loaded PLGA NPs

The effect of DOX-loaded PLGA NPs on the viability of EaRASMCs in vitro was assessed using a LIVE-DEAD® assay. As gauged by the absence of any dead cells in the fluorescence images, the DOX-PLGA NPs and the control 0% DOX-PLGA NPs were not cytotoxic at any of the provided concentrations.

DOX Release from PLGA NPs In Vitro

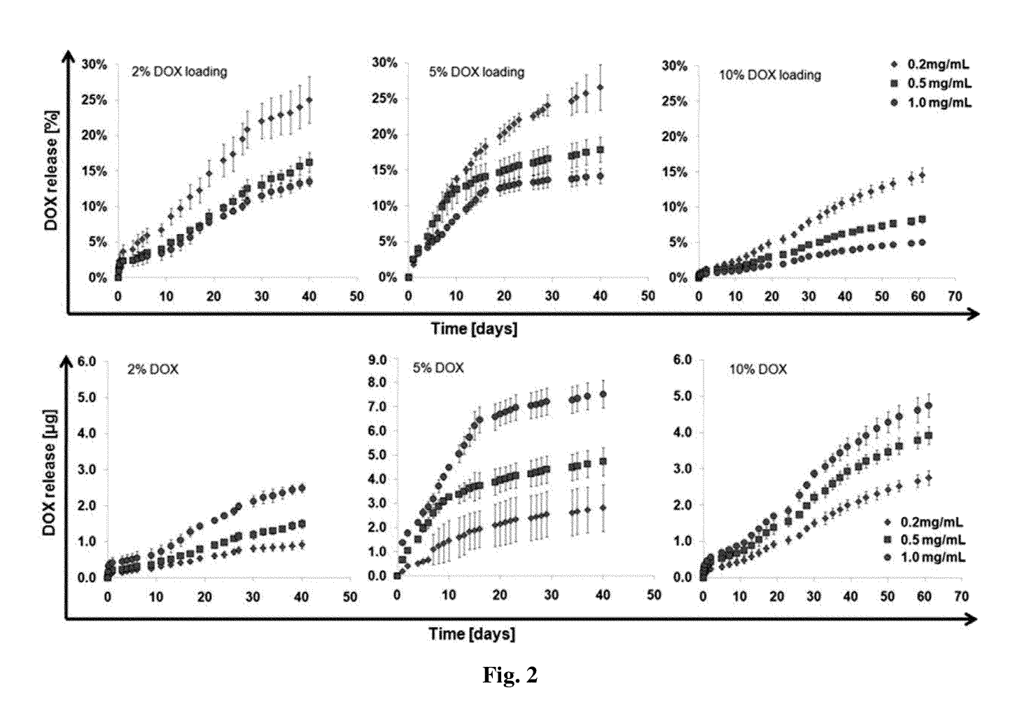

The in vitro release profiles for DOX from the PLGA NPs are shown in FIG. 2. Since DOX released at a 0.1 mg/mL concentration of NPs was close to the threshold of detection, release studies were performed with the higher NP concentrations of 0.2, 0.5 and 1.0 mg/mL. The DOX release curves obtained exhibit a characteristic biphasic release profile, characterized by an initial burst phase that spans the first day, followed by a slower exponential release phase similar to that observed for other drugs released from PLGA NPs. Panyam et al., J Control Release, 92:173-87 (2003).

The cumulative release of DOX over 40 days was less than 30% of the total amount of drug encapsulated for the 2 and 5% DOX-loaded PLGA NPs. The 10% DOX-loaded NPs on the other hand, demonstrated a lower percentage of cumulative release (3% to 11%) of the total DOX encapsulated, with the release continuing gradually up to 60 days.

Effects of DOX Released from NPs on SMC Proliferation and Elastic Matrix Synthesis

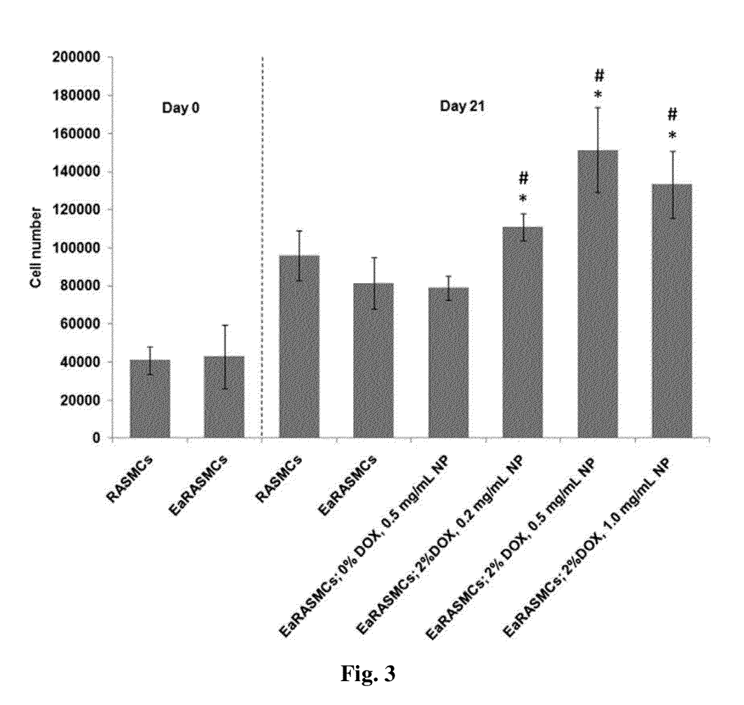

Based on previous studies by the inventors which demonstrated that an exogenous DOX dosage of 5.0 μg/mL limited the viability of aneurysmal RASMC cultures, 2% DOX loading condition were chosen to evaluate the functional effects of these DOX-loaded NPs on cell proliferation and elastic matrix synthesis, as the overall cumulative release over 21 days was below this threshold of 5 μg/mL. As observed in FIG. 3, the DOX released from the PLGA NPs did not inhibit EaRASMC proliferation. EaRASMC proliferation was found to be significantly higher (p<0.05) for the DOX NP treated cultures compared to the untreated and blank NP controls.

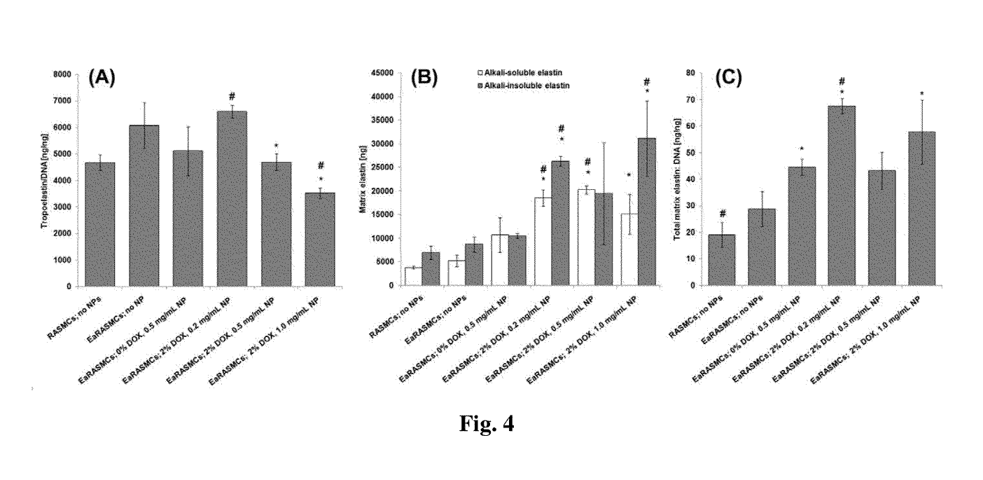

From an elastogenic standpoint, DOX released from the NPs did not affect tropoelastin (elastin precursor) synthesis on a per cell basis (FIG. 4A), as evidenced by the fact that there was no significant difference (p>0.05) between the values obtained for EaRASMC cultures treated with 2% DOX-loaded NPs at 0.2 mg/mL NP concentration and that of untreated EaRASMC cultures. Although we observed a decrease in tropoelastin production with increasing NP concentration of 2% DOX-loaded PLGA NPs, the inventors also observed that DOX released induced a significant increase in total matrix elastin deposition (on a per cell basis), relative to non-NP additive controls (FIG. 4C). Interestingly, EaRASMCs treated with 0% DOX-PLGA NPs also showed enhanced elastin matrix deposition compared to the non-additive controls. Nevertheless, increases in elastic matrix deposition induced by the DOX-loaded NPs was significantly greater (p<0.05) than that induced by the unloaded NPs.

Impact of DOX Released from PLGA NPs on MMP-2 Production and Activity in EaRASMC Cultures

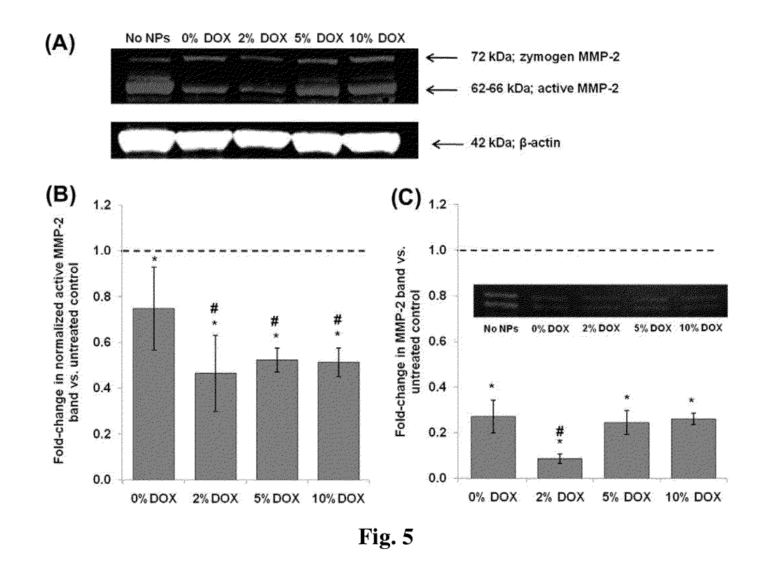

FIG. 5A shows a representative western blot for MMP-2 produced by EaRASMCs subject to the different NP-treatment conditions, along with the NP-untreated control. It must be noted that the intensity of the active MMP-2 band was normalized to the intensity of its corresponding β-actin band (loading control) for each test condition, in order to compare the effects between them. This normalized value for five biological replicates was plotted in FIG. 5B for each test case, and clearly shows that DOX released from the PLGA NPs is functional in causing an ˜50% inhibition of the active form of MMP-2, relative to the NP-untreated control. Surprisingly, even the 0% DOX-PLGA NPs caused a ˜25% decrease in expression of active MMP-2, and this was deemed to be statistically significant (p<0.05) compared to NP-untreated control. In all cases, MMP-9 bands were absent or too faint to be quantified reliably via densitometry.

Gel zymography data shown in FIG. 5C demonstrates that DOX-loaded NPs significantly inhibit (p<0.05) MMP-2 activity, with the samples treated with 2% DOX-PLGA NPs causing an almost 10-fold decrease. Interestingly, EaRASMCs cultured with 0% DOX-PLGA NPs caused a ˜4-fold attenuation in MMP-2 activity.

### Immunofluorescence Detection of Elastin and LOX

Confocal images for EaRASMC layers immunolabeled for elastin and LOX at the end of 21 days of culture with DOX-loaded PLGA NPs, as well as 0% DOX-PLGA NPs as active-agent controls and the treatment controls without any NPs, were obtained. The images of all culture groups show that there is significant deposition of elastin and LOX in the extracellular space surrounding the cells, and only limited elastin within cells. Although there was no apparent evidence of fiber formation, this is not entirely unexpected in a static, 2-D culture system, and it remains to be determined whether functional elastin fibers will be formed in vivo.

### Impact of Choice of Cationic Amphiphilic Surfactant on PLGA NP Characteristics

Although PLGA NPs were imparted with a positive surface charge by using 1% w/v DMAB directly as the stabilizer during the NP formulation process, similar use of 1% w/v DTAB and DAH as the stabilizers failed to generate any NPs. Therefore, PLGA NPs were first formulated using PVA as the stabilizer, and then functionalized with 1% w/v DTAB and DAH.

As shown in Table 2, PLGA NPs prepared using PVA as the surfactant were similar in diameter to those prepared with DMAB which had a diameter of 332.7±8.3 nm (see Table 1), but exhibited a strong negative surface charge, comparable to that obtained in an earlier study by Labhasetwar et al. (−27.8±0.5 mV). Labhasetwar et al., J Pharm Sci., 87:1229-34 (1998). Modification of these NPs with 1% w/v DTAB and DAH led to the neutralization of this strong negative charge on the NPs, without any significant alteration in NP size, as also indicated in Table 2. However, we have shown that it is possible to still formulate NPs using 5% w/v DAH directly as the surfactant and impart them with a positive surface charge, by increasing the DAH concentration.

TABLE 2
Size and surface charge of PLGA NPs formulated with PVA as surfactant,
and functionalized with DTAB and DAH. (n = 6, mean ± S.D.).

| | | 1% w/v PVA + | 1% w/v PVA + | 5% |
|---|---|---|---|---|
| Surfactant | 1% w/v PVA | 1% w/v DTAB | 1% w/v DAH | w/v DAH |
| Size [nm] | 336.0 ± 13.3 | 337.4 ± 15.6 | 330.6 ± 14.6 | 298.3 ± 26.9 |
| ζ-potential | −35.4 ± 3.6 | 1.3 ± 2.1 | 2.5 ± 4.4 | 20.1 ± 4.0 |
| [mV] | | | | |

### Binding of Modified PLGA NPs to Elastin

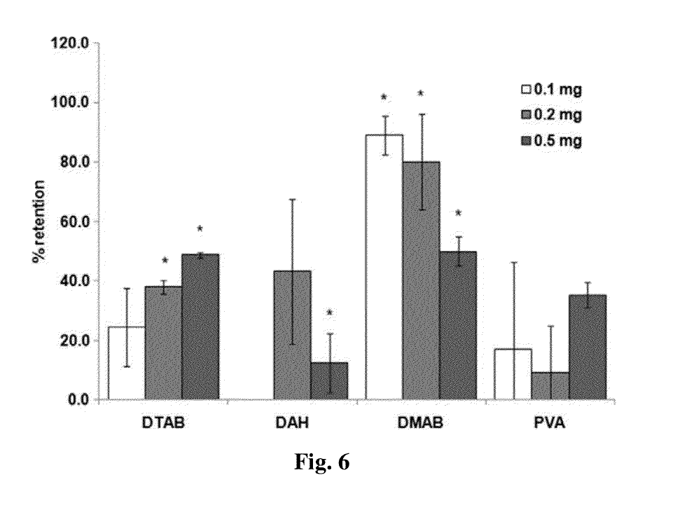

BSA-Cy5 encapsulated PLGA NPs functionalized with the different amphiphilic surface-modifiers were utilized to quantitatively compare their binding to elastin using a fluorometric method. As shown in Table 3, the sizes and zeta potential of the BSA-Cy5 loaded surface-modified PLGA NPs were comparable to unloaded (blank) surface-modified PLGA NPs listed in Table 2. Regardless of added amounts, PLGA NPs functionalized with DMAB demonstrated significantly greater binding to the elastin (p<0.05), compared to PVA-functionalized NPs, as seen in FIG. 6. Cationic DTAB-PLGA NPs also demonstrated enhanced elastin binding compared to negatively charged PVA-PLGA NPs, although their binding was lower than that observed for DMAB-PLGA NPs.

TABLE 3
Size and surface charge of PLGA NPs encapsulating BSA-Cy5,
formulated with PVA as surfactant, and functionalized with DTAB and
DMAB. (n = 6, mean ± SD).

| | 1% | 1% | 1% w/v PVA + | 1% w/v PVA + |
|---|---|---|---|---|
| Surfactant | w/v DMAB | w/v PVA | 1% w/v DTAB | 1% w/v DAH |
| Size [nm] | 275.0 ± 25.1 | 354.4 ± 19.0 | 382.9 ± 51.3 | 357.9 ± 56.7 |
| ζ-poten- | 25.6 ± 2.3 | −32.3 ± 3.6 | −0.4 ± 1.0 | 0.9 ± 0.5 |
| tial [mV] | | | | |

### Effect of Surface-Functionalized PLGA NPs on Lysyl Oxidase (LOX) Activity in SMC Cultures

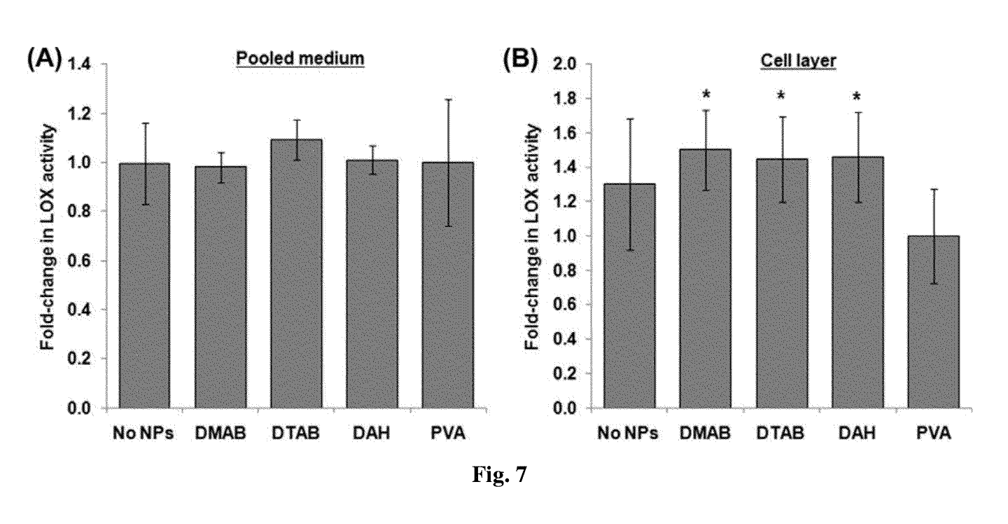

LOX activity in the EaRASMC cultures was found to be significantly enhanced (p<0.05) when treated with PLGA NPs which were surface-modified with the cationic amphiphiles DMAB, DTAB and DAH (FIG. 7B), relative to cultures treated with NPs that were surface-modified with PVA. Importantly, such increases were only detected in the cell layer fraction and not in the culture medium (FIG. 7A).

### Analysis of Effects of Cationic Surface Modification of NPs on MMP-2 Production and Activity

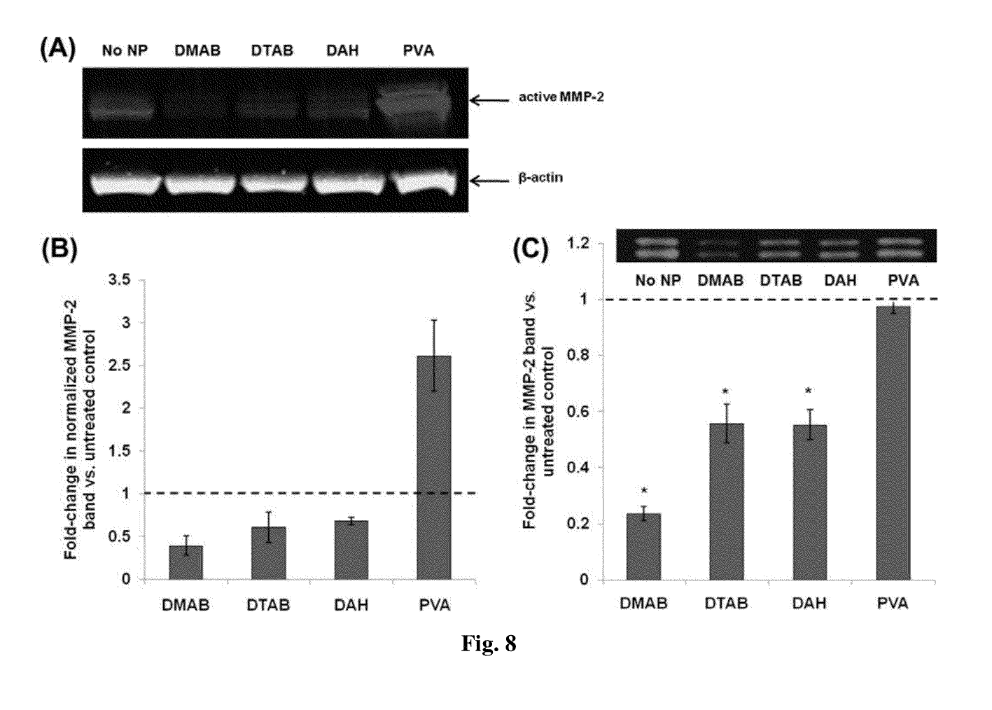

Based on the results obtained for the DOX-loaded NPs in EaRASMC cultures (FIG. 5), which demonstrated that the cationic surface charge on the NPs potentially plays a role in inhibiting MMP-2 production and activity, the effects of DOX-free DMAB-, DTAB-, DAH- and PVA-modified PLGA NPs on MMP-2 synthesis and activity were compared. The representative western blot for MMP-2 production by EaRASMCs for the different NP treatments (FIG. 8A) clearly illustrates that the functionalization of the DOX-free PLGA NPs with the cationic amphiphiles DMAB, DTAB and DAH, led to inhibition of active MMP-2 synthesis, while PVA functionalization appears to upregulate active MMP-2 production. Comparison of the β-actin normalized intensities of the MMP-2 bands for the different NP treatment conditions (FIG. 8B) demonstrated that the cationic functionalization of the PLGA NPs with DMAB led to a ˜2.5 fold downregulation of MMP-2 production compared to the NP-untreated control. The DTAB and DAH-functionalized NPs, both of which exhibit a near neutral mean ζ-potential, caused a ˜1.5 fold decrease in MMP-2 production compared to the NP-untreated control. On the other hand, the PVA-functionalized NPs, which exhibit a negative surface charge (mean ζ-potential=−32 mV), were found to cause a ˜2.5 fold enhancement of MMP-2 production compared to the NP-untreated controls. Results from gel zymograms shown in FIG. 8C confirm that the cationic surface charge on the NPs led to a significant inhibition of MMP-2 activity, as is illustrated by the ˜4 fold inhibition of MMP-2 activity by the DMAB-functionalized NPs. Although there was no significant difference in MMP-2 activity for the EaRASMC cultures treated with PVA-functionalized NPs compared to NP-untreated control cultures, both the DTAB- and DAH-functionalized NPs led to a 2-fold inhibition of MMP-2 activity.

### DISCUSSION

The overall goal of this study was to develop a NP-based delivery system for AAA therapy, which would enable localized, controlled, and sustained release of DOX over an extended duration (>3 weeks when ultimately delivered within AAA tissue), so as to avoid systemic action observed with oral DOX delivery. The inventors further sought to surface-functionalize these NPs with cationic amphiphiles to impart them with a positive surface charge, for potentially enhancing their arterial uptake, while improving their elastogenic benefits due to increased elastin binding and upregulation of elastin crosslinking by LOX.

PLGA has been approved by the FDA for drug delivery applications on account of its biodegradability, biocompatibility with a variety of drugs, appropriate biodegradation characteristics, as well as its ease of formulation. For this study NPs formulated with PLGA containing a 50:50 lactide:glycolide ratio was used, since it is the predominant form of PLGA used in nanotechnology applications including targeted, controlled drug delivery. Lue et al., Expert Rev Mol Diagn., 9:325-41 (2009). Additionally, the PLGA had an inherent viscosity of 0.95-1.2 dL/g (molecular weight equivalent to 117,700 Da, as specified by the company), as high molecular weight PLGA exhibits slower degradation compared to low molecular weight PLGA.

The inventors' studies indicated that NP sizes greater than 200 nm substantially remain in the extracellular space, with minimal intracellular uptake by EaRASMC. Although NPs of 500 nm size were almost completely excluded by the cells, polymeric NPs of this size and greater have been shown to undergo phagocytic uptake, which is undesirable. Nguyen et al., J Biomed Mater Res., 88A:1022-30 (2009). This suggests that an optimal NP size range for the proposed application lies between 200 and 500 nm. PLGA NPs formulated in this study with DMAB as the stabilizer were within this size range (mean hydrodynamic diameter=300-350 nm, as listed in Table 1). While NPs of this size exhibited some degree of internalization within EaRASMCs, this was significantly less pronounced than that observed with the 100 and 200 nm NPs. Further, the inventors observed considerable NPs continued to remain localized in the extracellular space and on top of the cells. Coupled with the observations that these NPs (a) significantly improved elastin crosslinking and other facets of elastic matrix deposition (discussed later in this section), which occur in the extracellular space, and (b) did not adversely impact cell viability, it may be concluded that NP sizes of ˜350 nm would likely be ideal for the proposed application. Further, DOX encapsulation within these NPs did not affect either their size or surface charge.

These NPs did not affect EaRASMC viability, demonstrating their non-cytotoxic nature. From the perspective of drug release from PLGA NPs, in some situations, the initial burst release phase during the first 24 h of release can be of concern, particularly from the aspect of the drug release exceeding toxic limits, and also from the inability of the formulation to provide sustained release of the agent(s). However, DOX released during the initial phase accounted for less than 5% of the total DOX encapsulated, and was less than 1.5 μg/mL, which was well below an exogenously delivered dose of 5.0 μg/mL that the inventors have observed to inhibit proliferation as well as elastic matrix synthesis by EaRASMCs. Hence, the burst release of DOX is likely not a concern. In the release curves described herein (FIG. 2), the DOX release from the 10% DOX-loaded NPs was lower than that observed for 5% DOX-loaded NPs. This could be explained by the inventors hypothesis that there exists a loading threshold (likely between 5 and 10% w/w DOX loading) beyond which DOX molecules are so compactly packed within the polymeric NPs, such that they hinder infiltration of aqueous medium, DOX solubilization and diffusion out. Thus, the 5% DOX-loaded NPs wherein DOX loading falls below the above defined threshold, are still be able to release DOX without any hindrance, which is higher (in terms of absolute amount) than the 2% DOX-loaded NPs. Regardless, the results clearly demonstrate the suitability of DOX-loaded PLGA NPs for controlled DOX release over a duration of more than 40 days, at a neutral (near physiologic) pH.

Standard oral doses of DOX greater than 30 mg/kg/day have been found to slow the growth of rat AAAs, but have also been shown to significantly inhibit SMC proliferation and migration, as well as decrease arterial elastin content in balloon-injured rat carotid arteries. Bendeck et al., Am J Pathol., 160:1089-95 (2002). The DOX concentrations equivalent to these oral doses as measured within AAA tissues in mice and in human patients has been shown to be ˜50 ng/mL, while levels in the circulation are between 2 and 10 μg/mL. Ding et al., Vascular, 13:290-7 (2005). DOX and other tetracyclines have also been found to inhibit cell proliferation, migration and matrix synthesis by vascular cells. Pinney et al., J Cardiovasc Pharmacol., 42:469-76 (2003). Exogenously delivered DOX doses in the range of 16-54 μg/mL have been shown to inhibit proliferation and migration of SMCs isolated from rat carotid arteries, and concurrently inhibit elastic matrix synthesis. Franco et al., Am J Pathol., 168:1697-709 (2006). In this present study, it was observed that EaRASMC proliferation was not inhibited (FIG. 3), as the DOX released from the 2% DOX-PLGA NPs was below the 5 μg/mL threshold which was found to limit the viability of rat aneurysmal SMCs in culture. In fact, it was found that the proliferation was significantly higher for DOX-NP treated EaRASMC cultures compared to those which received blank NPs, and also untreated controls. Therefore, based on the results with EaRASMCs, the inventors suggest that DOX may have biphasic effects on cell proliferation as a function of the delivered dose.

From an elastogenic perspective, although a decrease in tropoelastin production (normalized to cellular DNA content) was observed with increasing NP concentration for the DOX-loaded NPs (FIG. 4A), this could be attributed to (a) increased DOX release with increasing NP concentration potentially causing a decrease in tropoelastin production, (b) increased macromolecular crowding due to increasing NP concentration, or (c) both a & b. For example, recent studies have shown that macromolecular crowding within fibroblast cultures interfere with the release of procollagen (the collagen precursor molecule) into the cell culture medium, leading to enhanced collagen deposition in the cell layer. Chen et al., Adv Drug Deliv Rev., 63:277-90 (2011). However, it should also be noted that while tropoelastin is a key player in the elastic matrix deposition process, its levels in the cell culture medium may not be indicative of the extent of elastic matrix deposition. The pericellular matrix also plays a critical role in the formation and crosslinking of elastic fibers from tropoelastin molecules. Proteoglycans such as versican have been shown to impede elastic matrix assembly via binding of their negatively-charged chondroitin sulfate (CS) chains with the elastin binding protein (EBP), which leads to the premature release of EBP-bound tropoelastin molecules, preventing their interactions with the scaffold of microfibrils. Hinek A, Rabinovitch M., J Cell Biol., 126:563-74 (1994). The inventors hypothesize that increased elastic fiber assembly may occur as a result of positively charged NPs interacting with the negatively charged cell membrane of SMCs to form a sheath around, which in turn serves to shield the EBP from (negatively charged) CS, both by spatial exclusion and by electrostatic repulsion. As a result of this potential decrease/attenuation of CS/versican interactions with EBP, as well as their interactions with the crosslinking enzyme LOX which is negatively charged at physiological pH, the positively charged NPs may promote a microenvironment conducive for greater recruitment and crosslinking of tropoelastin precursors towards enhanced elastic matrix assembly.

Treatment with NPs caused an overall enhancement in elastic matrix deposition by EaRASMCs (as seen in FIGS. 4B and C), compared to NP-untreated healthy RASMCs and EaRASMC controls. Treatment with 0% DOX NPs significantly enhanced total matrix elastin on a per cell basis compared to untreated EaRASMCs, as seen in FIG. 4C, which may suggest that the macromolecular crowding effect due to the NPs, in addition to their positive surface charge, mediate enhanced elastic matrix deposition. A significant upregulation of elastic matrix deposition in EaRASMC cultures treated with 2% DOX-loaded NPs at 0.2 mg/mL NP concentration was also observed compared to untreated control cultures and cultures treated with 0% DOX-loaded NPs, which can be attributed to the effects of DOX. However, with an increase in NP concentration to 0.5 mg/mL, the normalized elastic matrix deposition in EaRASMC cultures treated with 2% DOX-loaded NPs was not significantly different from cultures treated with 0% DOX-loaded NPs at 0.5 mg/mL, suggesting that the DOX likely does not have any effect or may potentially be inhibitory towards elastic matrix deposition at this NP dose. However, upon closer analysis, it must be noted that the DOX released from the PLGA NPs significantly enhanced EaRASMC proliferation (FIG. 3) and total matrix elastin production (FIG. 4B), at all NP concentrations relative to the 0% DOX-loaded NPs at 0.5 mg/mL. Hence, the enhanced elastic matrix deposition observed for the treatment condition of 2% DOX-loaded NPs at 0.5 mg/mL was balanced by the concurrent increase in cell proliferation upon normalization of the matrix elastin to the cell number. This would potentially explain the lack of significant difference between the 0% and 2% DOX-loaded NPs at 0.5 mg/mL NP concentration. Based on this, it would be expected that 2% DOX-loaded NPs at an even higher NP concentration of 1.0 mg/mL would show lower elastic matrix content on a per cell basis. However, the macromolecular crowding effect mediated by the NPs likely offsets the effect of increased DOX concentration, leading to higher matrix elastin levels, and this concurs with FIG. 4A which showed lower tropoelastin in the medium for this treatment.

The surface charge and hydrophobicity of the NPs, on account of their functionalization with the cationic amphiphile DMAB likely play an important role in the enhanced elastic matrix deposition in the NP-treated EaRASMC cultures. The positive charge on the NPs potentially mediates LOX recruitment in the extracellular space via electrostatic interactions, as LOX is negatively charged at physiological pH. Additionally, as demonstrated in earlier studies with DTAB and DAH, which are chemical analogs of DMAB, one of the hydrophobic dodecyl chains of DMAB may bind to hydrophobic domains of tropoelastin molecules. Kagan et al., J Biol Chem., 256:5417-21 (1981). This facilitates the nucleation of these tropoelastin molecules and exposes their lysine side chains to lysyl oxidase (LOX), thereby enhancing their ability to be crosslinked into a matrix. Further, the net positive charge on the elastin-surfactant complex may also serve to improve or preserve the quality of newly deposited or pre-existing elastic matrix, and enhance the net accumulation of new elastic matrix by repulsion of elastase, which exhibits a net positive charge at physiological pH. Kagan et al., Biochem J., 177:203-14 (1979). Thus, the overall enhanced elastic matrix deposition observed in these studies can be attributed to (i) the charge and hydrophobicity of the NPs due to their surface functionalization with DMAB, as well as (ii) the macromolecular crowding effect due to the presence of these NPs in proximity to the cells, and (iii) the effects of DOX released from the NPs. It must be noted that the primary goal of the studies carried out for this manuscript was to examine the effects of DOX released from NPs on elastic matrix stabilization and (re)generation in EaRASMC cultures, with greater focus on the combinatorial effects of the DOX along with the NPs, rather than delineating their individual effects. Based on these results, the inventors propose to delineate the roles of these three critical parameters in mediating elastic matrix synthesis in SMC cultures in future studies.

DOX has been shown to be effective in suppressing aneurysmal dilatation in animal models of AAAs, as well as in humans by its inhibition elastolytic gelatinases MMP-2 and MMP-9 (Curci et al., J Vasc Surg., 31:325-41 (2000)), which are overexpressed within AAAs and contribute to their growth. Longo et al., J Clin Invest., 110:625-32 (2002). DOX-based inhibition of MMP production and activity is achieved via its chelation of the Zn²⁺ or Ca²⁺ ions in the catalytic domain of the active site of these enzymes (Galindo-Rodriguez et al., Pharm Res. 21:1428-39 (2004)), which causes their structural destabilization, leading to their denaturation and degradation. While the DOX released from the PLGA NPs was effective in inhibiting the synthesis and activity of MMP-2 (FIG. 5), the ability of DOX-free blank NPs to attenuate MMP-2 expression and activity was surprising. Based on these results, the inventors hypothesized that the ability of DOX-free PLGA NPs to inhibit MMP-2 synthesis and activity is due to their cationic surface charge. A recent study by Mendis et al. which showed the effects of a cationic glucosamine derivative in attenuating expression of MMP-2 and MMP-9 at the transcriptional level, while also inhibiting their activities, lends credence to this hypothesis. Mendis et al., Bioorg Med Chem Lett., 19:2755-9 (2009).

From a mechanism-based perspective, the results could be attributed to charge-based interactions of these cationic NPs with MMPs, particularly their active site(s). The catalytic domain of MMPs contains adjacent pairs of glutamic acid and histidine residues, which are essential for MMP activity, and mediate their binding to their substrates. At physiological pH, glutamic acid has a free carboxyl group, and thus remains negative charged. It has been hypothesized that cationic compounds, such as quarternary ammonium metacrylates, can bind to these glutamic acid residues via electrostatic interactions, to alter the configuration of the active site, and render it unrecognizable to complementary peptide sequences present in matrix proteins (e.g., elastin and collagen). Tezvergil-Mutluay et al., J Dent Res., 90:535-40 (2011). Additionally, they may also sterically prevent the active site from interacting with matrix proteins, thereby inhibiting MMP activity. Chlorhexidine, a cationic compound has also been shown to chelates cations to inhibit MMP-2 and -9 activity at lower doses, while at higher concentrations, it has been shown to inactivate MMP-2 by denaturation. Garcia et al., Mol Pharmacol., 67:1128-36 (2005). Similarly, cationic surfaces have also been shown to inhibit MMP-7 activity. Ganguly et al., FEBS Lett., 581:5723-6 (2007). The inventors hypothesize that the inhibitory effect of 0% DOX-PLGA NPs on MMP-2 activity may be due to their cationic surface charge. Their positive charge potentially enables them to bind to the negatively-charged glutamic acid residues in the catalytic domain of the active site of MMPs, leading to a decrease in their proteolytic activity. Further, on account of its large size, due to the presence of two dodecyl chains, it is also possible that DMAB may block the active site of MMP-2 via steric hindrance to exert its inhibitory effect. Thus, in addition to the effect of DOX released from the PLGA NPs, the cationic surface functionalization of the NPs with DMAB likely plays an important role in mediating the inhibition of MMP-2 activity.

DAH- and DTAB-functionalized PLGA NPs were formulated to compare their functional effects with those of DMAB-functionalized NPs in binding to elastin and also mediating LOX activity in EaRASMC cultures. Although DTAB- and DAH-modified NPs do not possess the high positive surface charge observed with DMAB-functionalized PLGA NPs, the long hydrophobic chains of DTAB and DAH may bind to the hydrophobic domains in elastin and ‘anchor’ the NPs to elastin/elastic fibers. The enhanced binding observed with DMAB-PLGA NPs (FIG. 6) can be attributed to the fact that it has the greatest hydrophobicity of the surfactants used. Esumi et al., Langmuir, 12:2130-5 (1996). Additionally, its binding capacity may also be enhanced due to the presence of two dodecyl chains in its structure, unlike DTAB and DAH, which have only a single dodecyl chain. PVA on the other hand, is a hydrophilic polymer which is used as a surfactant on account of its amphiphilic nature. Further, the surface charge on these NPs may also mediate their binding to elastin, as is evidenced by the fact that PLGA NPs directly formulated with DMAB exhibit a stronger cationic surface charge compared to their counterparts which were formulated with PVA as the stabilizer and then functionalized with DTAB or DAH (Tables 1 and 2). These results demonstrate the improved elastin binding capabilities achieved upon surface functionalization of PLGA NPs with cationic amphiphiles.

Based on the results with 5% w/v DAH leading to the formation of NPs with improved cationic surface charge, the inventors expect that increasing DTAB or DAH concentrations during NP formulation would lead to the formation of more positively-charged NPs which would potentially stimulate enhanced elastic matrix deposition and elastin binding, to an extent as observed for DMAB. On the other hand, increasing the DMAB concentration would likely lead to its saturation on the NP surface, with steric hindrance of the dodecyl chains on adjacent molecules, causing no further enhancement of elastin binding or matrix deposition. However, it must be noted that other factors such as DOX release and macromolecular crowding by the NPs mediate elastic matrix deposition.

Cationic PLGA NPs also exhibit enhanced binding interactions with SMCs. Panyam J, Labhasetwar V., Mol Pharm., 1:77-84 (2004). This is primarily on account of SMCs exhibiting a net negative charge (resting membrane potential=−55 mV). Further, since LOX is a negative charged enzyme at physiological pH, it is likely to bind via electrostatic interactions with the positively-charged PLGA NPs that would be expected to be attached similarly to the cell surface of SMCs. This is a potential explanation for the significantly higher levels of LOX activity observed for the cationic PLGA NPs modified with DMAB, DTAB and DAH, compared to the negatively-charged PVA-modified PLGA NPs (as seen in FIG. 7B). Electrostatic repulsion of the negatively-charged LOX molecules by the negatively-charged PVA-modified NPs would thus explain the decreased LOX activity. Further, the enhanced LOX activity observed in the cultures treated with cationic NPs also likely illustrate that the effect of their positive surface charge is maintained over the 21 day duration of the culture. Thus, surface modification of PLGA NPs using cationic amphiphiles improves LOX activity in EaRASMC cultures.

Additionally, the inventors demonstrated the ability of the DMAB-, DTAB-, and DAH-functionalized NPs to inhibit MMP-2 expression and activity in EaRASMC cultures (FIG. 8), compared to untreated control cultures. DMAB-functionalized NPs exhibited a greater extent of inhibition of MMP-2 expression and activity compared to DTAB- and DAH-functionalized NPs, which could be attributed to the higher cationic surface charge on the DMAB NPs. The fact that negatively-charged PVA-functionalized PLGA NPs caused a significant upregulation of MMP-2 expression, without any significant inhibition of its activity, confirms the critical role of the cationic surface charge on these NPs in attenuating MMP-2 expression and activity. However, at the same time, it must be noted that other studies have reported decreased MMP activity due to increased repulsion of negatively charged drug-conjugates by the negatively-charged active site of MMP-2. Chau et al., Bioconjug Chem., 15:931-41 (2004). Thus, future studies should examine the exact nature of the mechanisms underlying charge-based interactions in the attenuation of MMP activity.

### CONCLUSIONS

The results demonstrate the efficacy of controlled, long-term release of DOX from cationic PLGA NPs in inhibiting MMP-2 production and activity in EaRASMC cultures. Further, the DOX released did not inhibit cell proliferation and contrary to expectations, enhanced elastic matrix synthesis and accumulation. Along with the effects of the cationic surface charge in attenuating MMP-2 production and activity, these positively-charged NPs were also demonstrated to bind strongly to elastin, and also improve LOX activity in EaRASMC cultures. Thus, the cationic functionalization of these NPs clearly imparts them with a multifunctional nature, particularly in the context of the preservation and stabilization of elastic matrix towards regenerative repair in AAAs. The effects/benefits of the NP surface charge (and hydrophobicity), as well as the potential macromolecular crowding effect(s) from the pro-elastogenic standpoint warrant further examination in future work. Further studies will evaluate and optimize the delivery and retention of DOX-loaded PLGA NPs in vivo in animal models of AAA, as well as examine their functional effects in inhibiting matrix proteolysis and enhancing elastin regenerative repair, to stabilize the condition. This system may potentially be combined with endovascular repair techniques to develop a potent therapeutic strategy for stabilizing or even regressing AAAs. Overall, considering their potential ability to inhibit MMP and their enhanced ability for binding to elastin, these results demonstrate the multifunctional potential and utility of these cationic NPs in mediating improved elastogenic outcomes in aneurysmal SMC cultures.

### Example 2

### Preparation and Use of DOX-Encapsulating Superparamagnetic Iron Oxide Nanoparticles

Although DMAB-functionalization of the NP surface imparts a net positive surface charge that improves NP arterial uptake & retention, the inventors sought to incorporate superparamagnetic iron oxide NPs (SPIONs) within these cationic polymeric NPs towards further enhancing their uptake and localization within the AAA wall, upon guidance to the site using an applied external magnetic field. The physical size and surface charge of NPs containing DOX alone and co-encapsulated DOX & SPIONs were compared, as was DOX release from both sets of NPs to determine if SPION incorporation within the NPs altered any of these parameters. The volume fraction of magnetite within the NP formulations was determined to confirm successful incorporation of SPIONs. The magnetite volume fraction within the NPs also served as a metric for the extent of SPION loading. The velocity of the NP formulations under applied magnetic field was determined to ascertain their magnetic mobility. Further, their effects on the proliferation of rat AAA smooth muscle cell (EaRASMCs) cultures were examined in vitro. Finally, their effects on elastic matrix synthesis & MMP production and activity over 21 days were examined in vitro in EaRASMC cultures.

### Methods

Formulation and Characterization of DOX NPs and SPION-DOX NPs.

PLGA (50:50 lactide:glycolide) NPs were formulated via double-emulsion solvent evaporation method, with didodecyldimethylammonium bromide (DMAB) as the stabilizer. DMAB imparts NPs with a positive charge. NPs formulated were blank (no DOX or SPIONs), DOX-loaded (2% w/w DOX to PLGA), and (DOX+SPION)-loaded (2% w/w DOX, aminosilane-functionalized SPIONs (50 nm size) suspended at a concentration of 1 mg of SPIONs/100 μl of DOX solution). The size and surface charge (ζ-potential) of the composite NPs were determined via phase analysis light scattering. UV spectrophotometry (λ=270 nm) was used to determine the DOX encapsulation efficiency. Velocity of NPs under an applied magnetic field (0.105 T; magnetic gradient 0.008 T/mm) was determined using cell tracking velocimetry. (McCloskey et al., Anal Chem., 75: 6868-74 (2003)).

DOX Release from NPs

DOX release from the DOX-NPs and SPION-DOX NPs was carried out in phosphate buffer saline (PBS, pH 7.4; Sigma-Aldrich) at 37° C. on a shaker at 100 rpm. Briefly, 1.5 mL polypropylene microcentrifuge tubes (n=3 per formulation) were filled with 1.0 mL of NP suspensions containing 0.5 mg/mL of NPs. Release in each case, was carried out over 18 days. At each analysis time point, the samples were centrifuged (14,000 rpm, 30 min, 4° C.) in a microcentrifuge (Beckman Microfuge 16®, Beckman Coulter, Inc.), the supernatants withdrawn to quantify DOX content, and volume-replenished with fresh PBS. DOX released was quantified by UV-spectrophotometry (SpectraMax M2, Molecular Devices, Inc., Sunnyvale, Calif.). DOX absorbance at 270 nm, was calibrated to its concentration using serial dilutions of a 1.0 mg/mL DOX solution. The DOX standards were incubated under the same conditions as the NP samples, to avoid any time- and temperature-dependent degradation of DOX.

Experimental Design for Cell Culture

For cell culture experiments intended to investigate the impact of Blank PLGA, DOX-NPs, and SPION-DOX-NPs on cellular matrix synthesis, EaRASMCs (passages 2-5) were seeded at 3×10⁴ cells/well and cultured over 21 days in 6-well plates (A=10 cm²; BD-Biosciences, Franklin Lakes, N.J.). The cells were cultured in DMEM-F12 supplemented with 2% v/v FBS, 1% v/v PenStrep and 100 ng/mL TNF-α (PeproTech, Inc, Rocky Hill, N.J.) to simulate an aneurysmal or activated microenvironment in culture. NPs loaded with 2% w/w of DOX or 2% w/w of DOX and SPIONs were cultured with cell layers at concentrations of 0.1 mg/mL of NPs (n=6 per treatment) for 21 days. EaRASMCs cultured with DOX-unencapsulated NPs (blank NPs; 0.1 mg/mL of NPs) served as the active-agent (DOX) control. Healthy, passage-matched RASMCs were similarly seeded, without any NPs, as the cell type controls. Fresh medium was added to the cultures weekly, with the spent medium slowly pipetted out from individual wells pooled at each time point and stored at −20° C. The NPs were almost all bound to the cell layer, and were not removed along with the spent medium. The pooled spent medium aliquots were biochemically assayed along with their corresponding cell layers which were harvested after the 21 days of culture.

For western blots and zymography to compare the effect of DOX released from the DOX-NPs and SPION-DOX NPs on the proteolytic response, EaRASMCs were seeded at 2×10⁵ cells/well in a 6-well plate and cultured for 7 days in DMEM-F12 supplemented with 5% v/v FBS and 1% PenStrep, along with 100 ng/mL TNF-α. PLGA NPs (blank NPs, 2% w/w DOX-loaded NPs, and 2% w/w DOX and SPION-loaded NPs, each at 0.1 mg/mL of NPs) were added to the EaRASMCs at day 1 after seeding (n=3 per treatment).

DNA Assay for Cell Proliferation

The DNA content of the cell layers was measured via a fluorometric assay, to determine the effects of DOX released from the NPs on EaRASMC proliferation. The cell layers were harvested at 1 day and 21 days of culture in NaCl-Pi buffer, sonicated on ice and assayed for DNA content. The cell density was calculated, assuming 6 pg DNA/cell.

Fastin Assay for Elastin

The total elastin content in the cell layer (alkali-soluble and insoluble fractions), as well as the tropoelastin (soluble elastin precursor) released in the cell culture medium, was quantified using a Fastin assay (Accurate Scientific and Chemical, Westbury, N.Y. The cell layers were harvested after 21 days of culture, resuspended in NaCl-Pi buffer, and sonicated on ice to homogenize the cell layer. This suspension thus obtained was digested with 0.1N NaOH (1 h, 98° C.) and then centrifuged to yield a pellet containing mature, highly cross-linked alkali-insoluble elastin, with the supernatant fraction containing less cross-linked alkali-soluble elastin. The alkali-insoluble elastin was then converted into a soluble form prior to quantification, as the Fastin assay can only quantify soluble α-elastin. The pellet obtained after the NaOH digestion step was dried and solubilized with 0.25 M oxalic acid (1 h, 95° C.), after which they were pooled and centrifuge-filtered (3000 rpm, 10 min) in microcentrifuge tubes (Amicon® Ultra, 10 kDa molecular weight cut-off; Millipore, Inc., Billerica, Mass.). These soluble and insoluble matrix elastin fractions, as well as the tropoelastin fraction in the cell culture medium were then measured using the Fastin assay. The amounts of elastin measured were also normalized to their corresponding DNA amounts, so as to provide an accurate comparison between the different treatments.

Western Blots for MMP-2 and MMP-9 Protein Expression

MMP-2 and -9 production by activated EaRASMCs in the absence and presence of the PLGA NPs and NP-released DOX, with and without co-loaded SPIONs was semi-quantitatively assessed using western blots. After 7 days of culture, the cell layers were harvested in RIPA buffer (Thermo Scientific) containing Halt™ protease inhibitor (Thermo Scientific), and assayed for total protein content using a bicinchonic acid (BCA) assay kit (Thermo Scientific). Maximum volumes of sample protein (15.6 μL) were then loaded under reduced conditions into each lane of a 10% sodium dodecylsulfate-polyacrylamide gel electrophoresis (SDS-PAGE) gel (Invitrogen), along with a BenchMark™ pre-stained molecular weight ladder (Invitrogen). The gels were then transferred dry onto nitrocellulose membranes (iBlot® Western Blotting System, Invitrogen). The membranes were blocked with the Odyssey Blocking Buffer (LI-COR Biosciences, Lincoln, Nebr.) for 1 h, following which they were immunolabeled for 16 h at 4° C. with a rabbit polyclonal antibody against MMP-2 (1:500 dilution; Abcam, Cambridge, Mass.) and a rabbit monoclonal antibody against MMP-9 (1:500 dilution; Millipore, Inc.), with a mouse monoclonal antibody against β-actin (1:1000 dilution; Sigma-Aldrich) as the loading control. Secondary antibody labeling was carried out for 1 h at room temperature using IRDye® 680LT goat-anti-rabbit (1:15,000 dilution) and IRDye® 800CW goat-anti-mouse (1:20,000 dilution) polyclonal antibodies (LI-COR Biosciences). Fluoro-luminescent detection of the protein bands was then carried out using a LI-COR Odyssey laser-based scanning system. The intensities of active MMP-2 bands on all gels were quantified using ImageJ software, expressed in terms of relative density units (RDU) and normalized to the intensity of their respective β-actin bands to enable comparison between the different test cases within the same blot. The normalized band intensities of active MMP-2 for the PLGA NP-treated and other test cultures were further normalized to those of the NP-untreated control cultures to determine the fold-change(s) in production of active MMP-2, and the statistical significance of the differences between them. Results were averaged from 3 replicate gels run per culture treatment.

Gel Zymography for MMP-2 and MMP-9 Activity

Treatment-specific effects on cellular MMP-2 and -9 activities were analyzed via gel zymography. Briefly, volumes of the cell layer samples (harvested in RIPA buffer containing protease inhibitor) equivalent to 10 μg of protein, were loaded into each lane of a 10% zymogram gel (Invitrogen), along with a BenchMark™ pre-stained molecular weight ladder (Invitrogen). The gel was run for 2 h at 125 V. The gels were then washed in a buffer containing 2.5% v/v Triton-X-100 for 30 min to remove SDS, and then incubated overnight in a substrate/development buffer to activate the MMPs. The gels were stained with Coomassie Brilliant Blue solution for 45 min, and destained for 90 min, until clear bands appeared visible against the blue background of the gel. Band intensities (RDU) of the bands obtained for test cultures were measured using ImageJ software, and normalized to those obtained for the NP-untreated control cultures to determine fold changes in MMP-2 activity. Data was acquired from 3 independent replicate gels/treatment condition.

### Results

Table 4 shows data indicating that the physical properties (size and ζ-potential) of DMAB-surface functionalized PLGA NPs are not altered upon loading with DOX alone or with SPIONs. PLGA NPs functionalized with DMAB had mean sizes of 316 nm, with ζ-potential=+49 mV, and ˜40% DOX encapsulation efficiency (Table 4). Size and ζ-potential represent mean±SD (n=6). The data for magnetic properties represents mean±SEM for >500 NPs from 8 different tracking experiments. DOX encapsulation efficiency was ˜40% for both DOX and DOX-SPION NPs. SPION incorporation, confirmed by measurement of magnetitite volume (Table 4) did not affect NP size, surface charge or DOX encapsulation efficiency (Table 4). Unlike blank NPs and DOX NPs, the SPION-DOX NPs exhibited significant mobility even in the weak applied magnetic field (Table 4).

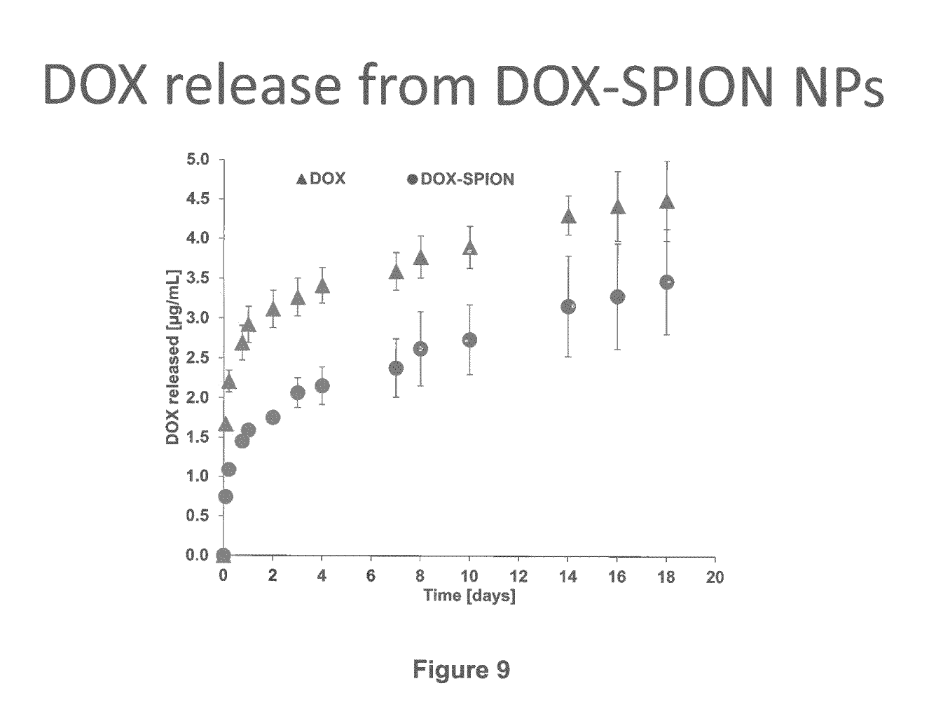

FIG. 9 shows that DOX release from DOX NPs ranged between 1-4.5 μg/mL over the ˜18 day period over which release was measured (FIG. 9), well below the 16-54 μg/mL range which has been shown to limit elastic matrix synthesis by SMCs. Over this entire period, DOX release from the SPION-DOX NPs was slightly lower than for DOX NPs. Considering that DOX loading efficiencies were maintained at 40%, the slower release is instead attributable due to tighter packing of DOX in presence of SPIONs and consequent lower permeability of aqueous medium for dissolution and effusion of the drug.

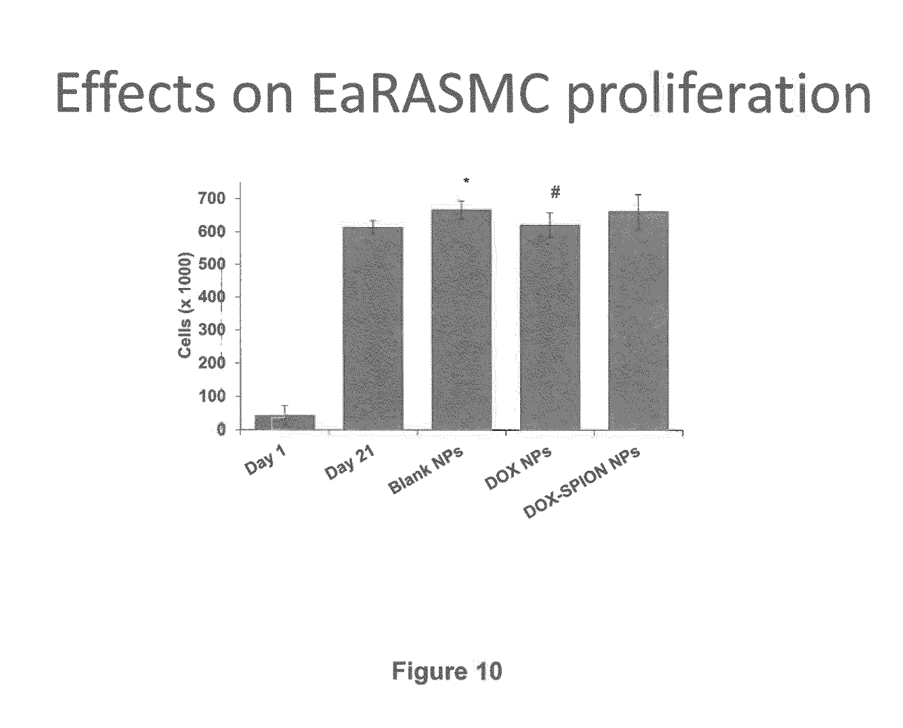

FIG. 10 provides a bar graph showing that the lack of effect of DOX released from DMAB-surface functionalized PLGA NPs on proliferation of rat aneurysmal smooth muscle cells (EaRASMCs), is maintained upon co-encapsulating SPIONs within the NPs.

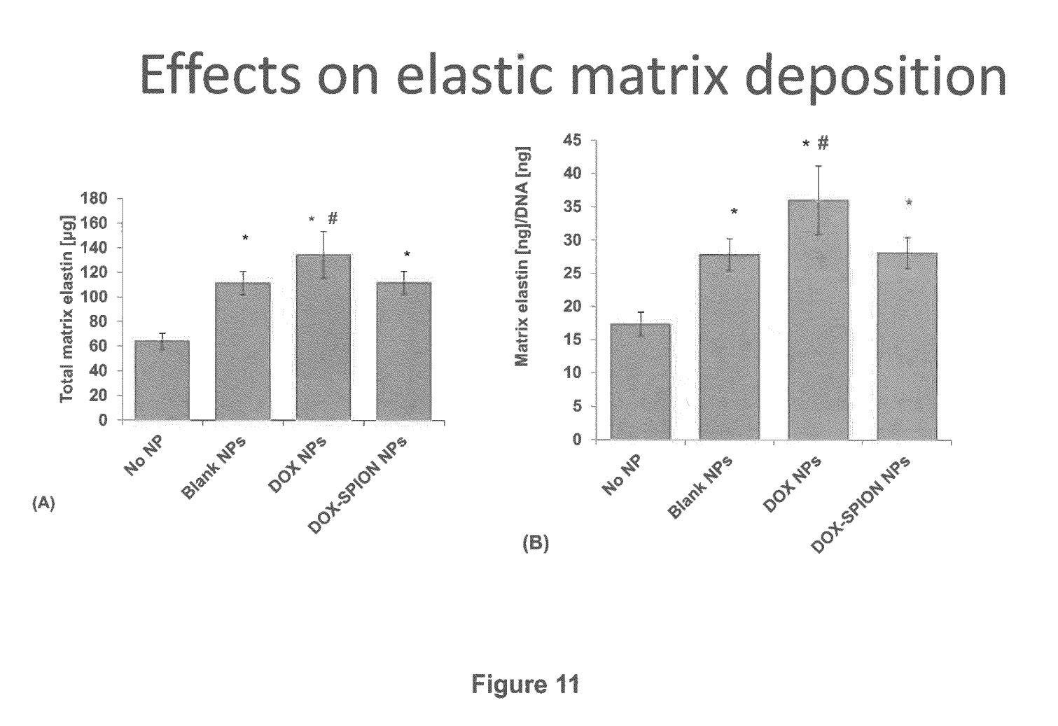

FIG. 11 indicates that cationic, DMAB functionalized NPs (all NP-treated cases) caused a significant improvement in elastic matrix deposition versus NP-free cultures. DOX release from the NPs further enhanced this effect. DOX release from NPs co-incorporating both DOX and SPIONs however did not induce similar further increases in elastic matrix deposition, likely due to slightly slower release of DOX in the presence of SPIONs (FIG. 9).

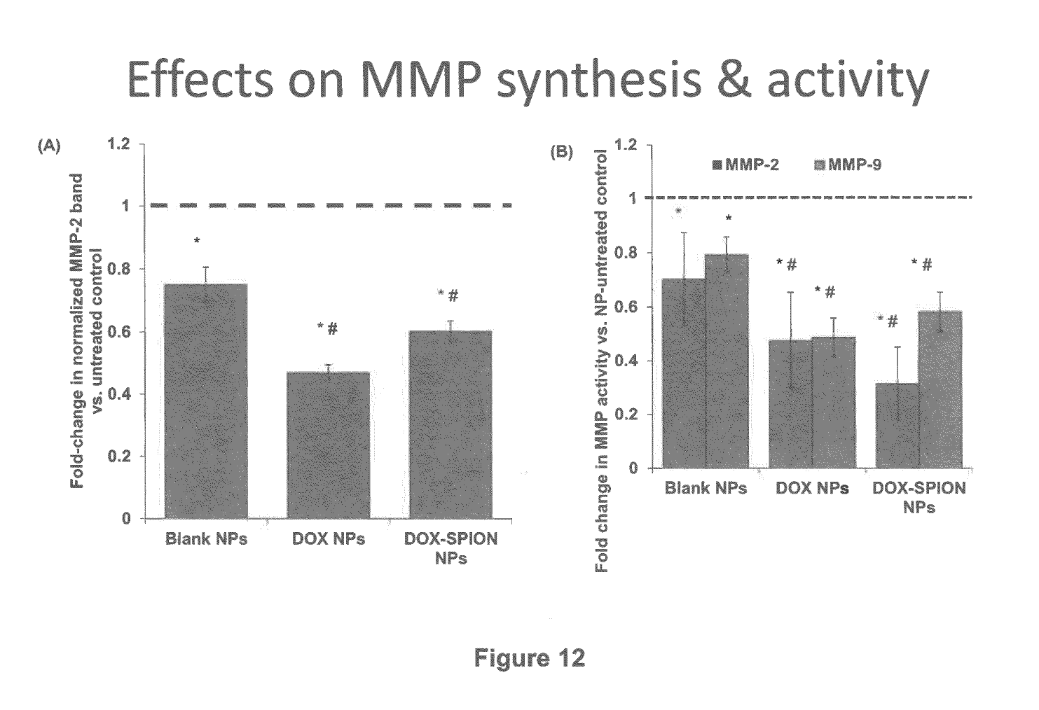

FIG. 12 shows significant decreases in MMP2 protein synthesis and enzyme activity in both EaRASMC cultures treated with DOX NPs and SPION-DOX NPs relative to NP-free cultures, and even versus blank NP-treated cultures. Despite the slightly lower release levels of DOX from the DOX-SPION NPs versus the DOX NPs, no significant differences in levels of attenuation of MMP2 (protein synthesis or enzyme activity) were noted in cultures treated with these respective NP formulations. MMP-9 protein synthesis was too low for reliable quantification via densitometry, and hence excluded.

TABLE 4
Physical and magnetic properties of PLGA NPs.

| | | | Magnetic | Volume |
|---|---|---|---|---|
| | Size | ζ-potential | velocity | fraction of |
| Sample | [nm] | [mV] | [μm/s] | magnetite [%] |
| Blank NPs | 316.1 ± 6.3 | 49.4 ± 7.0 | N/A | N/A |
| DOX NPs | 360.8 ± 5.1 | 51.7 ± 4.5 | N/A | N/A |
| DOX- | 319.2 ± 11.7 | 49.6 ± 3.0 | 1.90 ± 0.02 | 12.8 ± 0.1 |
| SPION NPs | | | | |

Co-incorporation of DOX along with SPIONs caused a significant decrease in magnetic velocity, as well as the volume fraction of magnetite within the NPs. SPION-loaded DMAB-NPs exhibited higher velocities due to higher volume fraction of magnetite encapsulated within. In vitro and in vivo studies will provide additional insights into the feasibility of this strategy as a modality for enhancing the targeting efficacy of NPs for localized, controlled and sustained therapeutic delivery at the AAA site.

The complete disclosure of all patents, patent applications, and publications, and electronically available materials cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. An elastogenic nanoparticle comprising a polymeric core having a surface that is functionalized with a cationic amphiphilic compound, and comprising an active agent having pro-elastogenic and/or anti-proteolytic activity.

2. The elastogenic nanoparticle of claim 1, wherein the polymeric core comprises poly(lactic-co-glycolic acid).

3. The elastogenic nanoparticle of claim 1, wherein the active agent is an anti-proteolytic agent.

4. The elastogenic nanoparticle of claim 1, wherein the active agent is a matrix metalloproteinase inhibitor.

5. The elastogenic nanoparticle of claim 3, wherein the matrix metalloproteinase inhibitor is doxycycline.

6. The elastogenic nanoparticle of claim 1, wherein the active agent is dispersed within the polymeric core of the nanoparticle.

7. The elastogenic nanoparticle of claim 1, wherein the active agent is linked to the surface of the nanoparticle via a proteolytically-sensitive peptide linkage.

8. The elastogenic nanoparticle of claim 1, wherein an imaging agent is linked to the surface of the nanoparticle via a proteolytically-sensitive peptide linkage.

9. The elastogenic nanoparticle of claim 1, wherein the cationic amphiphilic compound is didodecyldimethyl ammonium bromide.

10. The elastogenic nanoparticle of claim 1, wherein the particle has a diameter from about 300 to about 500 nanometers.

11. The elastogenic nanoparticle of claim 1, wherein the particle has a surface charge from about +10 mV to about +50 mV.

12. The elastogenic nanoparticle of claim 1, wherein the particles further comprise a superparamagnetic iron oxide.

13. A method of stimulating elastogenesis in a subject by administering to the subject a therapeutically effective amount of elastogenic nanoparticles comprising a polymeric core having a surface that is functionalized with a cationic amphiphilic compound.

14. The method of claim 13, wherein the polymeric core of the elastogenic nanoparticles comprise poly(lactic-co-glycolic acid).

15. The method of claim 13, wherein the elastogenic nanoparticles further comprise an active agent having pro-elastogenic and/or anti-proteolytic activity.

16. The method of claim 15, wherein the active agent is an anti-proteolytic agent.

17. The method of claim 15, wherein the active agent is a matrix metalloproteinase inhibitor

18. The method of claim 17, wherein the matrix metalloproteinase inhibitor is doxycycline.

19. The method of claim 13, wherein the active agent is dispersed within the polymeric core.

20. The method of claim 13, wherein the cationic amphiphilic compound is didodecyldimethyl ammonium bromide.

21. The method of claim 13, wherein the elastogenic nanoparticle has a diameter from about 300 to about 500 nanometers.

22. The method of claim 13, wherein the subject has been diagnosed as having an abdominal aortic aneurysm.

23. The method of claim 13, wherein the elastogenic nanoparticles are delivered in a pharmaceutically acceptable carrier.

24. The method of claim 13, wherein the subject has periodontal disease.

25. The method of claim 13, wherein the elastogenic nanoparticles further comprise a superparamagnetic iron oxide, and the method further comprises directing elastogenic nanoparticles that have been administered to the subject using a magnetic field.

26. A pharmaceutical formulation comprising elastogenic nanoparticles and a pharmaceutically acceptable carrier, wherein the elastogenic nanoparticles comprise a polymeric core having a surface that is functionalized with a cationic amphiphilic compound, and comprising an active agent having pro-elastogenic and/or anti-proteolytic activity.

27. The pharmaceutical formulation of claim 26, wherein the active agent is an anti-proteolytic agent.

28. The pharmaceutical formulation of claim 26, wherein the active agent is a matrix metalloproteinase inhibitor.

29. The pharmaceutical formulation of claim 26, wherein the active agent is dispersed within the polymeric core.

30. The pharmaceutical formulation of claim 26, wherein the formulation is a topical formulation.

31. The pharmaceutical formulation of claim 26, wherein formulation is a parenteral formulation.

32. The pharmaceutical formulation of claim 26, wherein the formulation coats the surface of or is admixed within a biocompatible scaffold.

33. The pharmaceutical formulation of claim 26, wherein the elastogenic nanoparticles further comprise a superparamagnetic iron oxide.
